(19) United States
(12) Patent Application Publication
(10) Pub. No.: US 2025/0053237 A1
(43) Pub. Date: Feb. 13, 2025

(54) DEVICES, SYSTEMS, AND METHODS FOR CHARACTERIZING MOTIONS OF A USER VIA WEARABLE ARTICLES WITH FLEXIBLE CIRCUITS

(71) Applicant: Liquid Wire Inc., Portland, OR (US)

(72) Inventors: Trevor Antonio Rivera, Portland, OR (US); Jorge E. Carbo, JR., Portland, OR (US); Mark William Ronay, Portland, OR (US); Michael Adventure Hopkins, Portland, OR (US); Katherine M. Nelson, Portland, OR (US); Iheanyi Eronini Marike, Portland, OR (US); Jerry Gene Sewell, JR., Portland, OR (US); Michael Scott Amos, Beaverton, OR (US)

(73) Assignee: Liquid Wire Inc., Portland, OR (US)

(21) Appl. No.: 18/806,448

(22) Filed: Aug. 15, 2024

Related U.S. Application Data
(63) Continuation of application No. PCT/US23/62668, filed on Feb. 15, 2023.
(60) Provisional application No. 63/268,063, filed on Feb. 15, 2022.

(51) Int. Cl. G06F 3/01; G06F 1/16

(52) U.S. Cl. CPC G06F 3/014; G06F 1/1694

(57) ABSTRACT

A system configured to characterize a physical motion performed by a user is disclosed herein. The system can include a wearable article including a first flexible circuit, that includes a first trace formed from a deformable conductor. The first flexible circuit is positioned in a first location of interest on the wearable article. The system can further include a computing device configured to receive a first signal generated by the first flexible circuit, determine a first electrical parameter based on the first signal, determine a physical condition of the first flexible circuit based on the first electrical parameter, compare the physical condition of the first flexible circuit to previously determined physical conditions associated with the wearable article, and characterize the physical motion performed by the user based on the comparison.

### CROSS-REFERENCE TO RELATED APPLICATIONS

The present application is a continuation of and claims the benefit of priority from PCT/US23/62668, filed on 15 Feb. 2023, which claims the benefit of priority from U.S. Provisional Patent Application No. 63/268,063 filed Feb. 15, 2022. The disclosures of both applications are herein incorporated by reference in their entireties.

### FIELD

The present disclosure is generally related to flexible circuits and, more particularly, is directed to flexible circuits that can be integrated into wearable articles for the purposes of physical motions in a real environment.

### SUMMARY

The following summary is provided to facilitate an understanding of some of the innovative features unique to the aspects disclosed herein and is not intended to be a full description. A full appreciation of the various aspects can be gained by taking the entire specification, claims, and abstract as a whole.

In various aspects, a system configured to characterize a physical motion performed by a user is disclosed. The system can include a wearable article including a first flexible circuit that includes a first trace formed from a deformable conductor. The first flexible circuit is positioned in a first location of interest on the wearable article. The system can further include a computing device configured to receive a first signal generated by the first flexible circuit, determine a first electrical parameter based on the first signal, determine a physical condition of the first flexible circuit based on the first electrical parameter, compare the physical condition of the first flexible circuit to previously determined physical conditions associated with the wearable article, and characterize the physical motion performed by the user based on the comparison.

In various aspects, a system configured to simulate a physical motion performed by a user via an avatar in a virtual environment is disclosed. The system can include: a wearable article communicably including a first flexible circuit, wherein the first flexible circuit includes a first trace including a deformable conductor, and wherein the first flexible circuit is positioned in a first location of interest on the glove. The system can further include a computing device including a processor and a memory configured to store a visualization engine that, when executed by the processor, causes the processor to: receive a first signal from the first flexible circuit; determine a first electrical parameter based on the first signal; scale the first electrical parameter based on a predetermined simulation framework of the visualization engine, wherein scaling the first electrical parameter corresponds to a physical condition of the first flexible circuit; compare the physical condition of the first flexible circuit to previously determined physical conditions associated with the wearable article; and generate a simulation of the physical motion performed by a user via the avatar in the virtual environment based on the comparison.

In various aspects, a wearable article configured to simulate a physical motion performed by a user via an avatar in a virtual environment is disclosed herein. The wearable article can include a first flexible circuit, wherein the first flexible circuit includes a first trace including a deformable conductor, wherein the first flexible circuit is positioned in a first location of interest on the wearable article. The wearable article can further include a circuit configured to communicably couple the first flexible circuit to a computing device, including a processor and a memory configured to store a visualization engine that, when executed by the processor, causes the processor to: receive a first signal generated by the first flexible circuit; determine a first electrical parameter based on the first signal; scale the first electrical parameter based on a predetermined simulation framework of the visualization engine, wherein scaling the first electrical parameter corresponds to a physical condition of the first flexible circuit; compare the physical condition of the first flexible circuit to previously determined physical conditions associated with the wearable article; and generate a simulation of the physical motion performed by a user via the avatar in the virtual environment based on the comparison.

In various aspects, a method of simulating a physical motion performed by a user via an avatar in a virtual environment is disclosed. The method can include: developing a framework for electrical parameters generated by a plurality of flexible circuits of a wearable article, wherein the framework includes a plurality of scales that correlate the electrical parameters generated by each flexible circuit of the plurality of flexible circuits to a plurality of physical conditions of each flexible circuit of the plurality of flexible circuits; receiving a plurality of signals generated in response to a user's motions while wearing the wearable article, wherein the plurality of signals correspond to electrical parameters generated by the plurality of flexible circuits of the wearable article; determining a first physical condition of a first flexible circuit of the plurality of flexible circuits based on a first received signal of the plurality of signals and the plurality of scales; determining a second physical condition of a second flexible circuit of the plurality of flexible circuits based on a second received signal of the plurality of signals and the plurality of scales; comparing the first physical condition to the second physical condition; and generating a simulation of the physical motion performed by a user via the avatar in the virtual environment based on the comparison.

In various aspects, a glove configured to generate a virtual representation of a physical motion performed by a user of the glove is disclosed. The glove can include a first flexible circuit including a first trace including a deformable conductor and a first electrical feature electrically coupled to the trace, wherein the first electrical feature is positioned in a first location of interest on the glove, and a second flexible circuit including: a second trace including a deformable conductor and a second electrical feature electrically coupled to the trace, wherein the second electrical feature is positioned in a second location of interest on the glove, and wherein the glove is configured to be communicably coupled to a processor and a memory configured to store instructions that, when executed by the processor, cause the processor to: receive a first signal from the first flexible circuit; determine a first electrical parameter based on the first signal; correlate the first electrical parameter to a first physical parameter associated with the first location of interest; receive a second signal from the second flexible circuit; determine a second electrical parameter based on the second signal; correlate the second electrical parameter to a second physical parameter associated with the second location of interest; compare the first physical parameter associated with the first location of interest to the second physical parameter associated with the second location of interest; and generate the virtual representation of the physical motion performed by the user of the glove based on the comparison of the first physical parameter associated with the first location of interest to the second physical parameter associated with the second location of interest.

In various aspects, a method of generating a virtual representation of a physical motion performed by a user of a glove including a plurality of flexible circuits is disclosed. The method can include: performing a first motion while wearing the glove; generating, via a first flexible circuit of the plurality of flexible circuits, a first electrical parameter associated with the first motion; generating, via a camera, motion capture data associated with the performance of the first motion; correlating, via a processor communicably coupled to the glove, the generated motion capture data to the generated first electrical parameter; storing, via a memory communicably coupled to the processor, the correlation; repeating the first motion while wearing the glove; and generating, via the processor, a virtual replication of the first motion based exclusively on the stored correlation of the generated motion capture data to the generated first electrical parameter.

These and other features and characteristics of the present disclosure, as well as the methods of operation, functions of the related elements of structure, and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Various features of the aspects described herein are set forth with particularity in the appended claims. The various aspects, however, both as to organization and methods of operation, together with advantages thereof, may be understood in accordance with the following description taken in conjunction with the accompanying drawings as follows:

FIG. 1 illustrates a strain sensor system including a two-dimensional strain sensor, according to at least one non-limiting aspect of the present disclosure;

FIGS. 2A-E illustrate individual layers of a medium of the strain sensor system of FIG. 1, according to at least one non-limiting aspect of the present disclosure;

FIGS. 3A and 3B illustrate traces of a strain sensor system in a relaxed condition and a deformed condition, according to at least one non-limiting aspect of the present disclosure;

FIG. 4 illustrates another strain sensor, according to at least one non-limiting aspect of the present disclosure;

FIGS. 5-11 illustrate various electrodes that can be implemented via the wearable articles disclosed herein, according to at least one non-limiting aspect of the present disclosure;

FIGS. 12-14 illustrate various sleeves from which the wearable articles disclosed herein can be formed, according to at least one non-limiting aspect of the present disclosure;

FIG. 15 illustrates a circuit configured for use with any of the strain sensors, electrodes, and articles disclosed herein, according to at least one non-limiting aspect of the present disclosure;

FIG. 16 illustrates a method of correlating data generated by a strain gauge sensor to data generated by an inertial measurement unit (“IMU”) data, according to at least one non-limiting aspect of the present disclosure;

FIGS. 17 and 18 illustrate another flexible circuit configured for use with the articles disclosed herein, according to at least one non-limiting aspect of the present disclosure;

FIG. 19 illustrates an article configured to track physical motions of a user, according to at least one non-limiting aspect of the present disclosure;

FIG. 20 illustrates one of the substrates of the glove of FIG. 19 depicted in FIG. 21, according to at least one non-limiting aspect of the present disclosure;

FIG. 21 illustrates another article configured to track physical motions of a user, according to at least one non-limiting aspect of the present disclosure;

FIG. 22 illustrates another substrate configured for use with another glove, according to at least one non-limiting aspect of the present disclosure;

FIG. 23 illustrates a method of generating signals associated with electrical parameters and correlating those electrical parameters to the physical motions of a user of the gloves, according to at least one non-limiting aspect of the present disclosure;

FIGS. 24A and 24B illustrate the glove of FIG. 21 in use via the method of FIG. 23, according to at least one non-limiting aspect of the present disclosure;

FIG. 25 illustrates a system configured to simulate motions in a virtual environment using a wearable article with flexible circuits, according to at least one non-limiting aspect of the present disclosure;

FIGS. 26A-C illustrate a simulation framework configured to be run via the system of FIG. 25, according to at least one non-limiting aspect of the present disclosure;

FIG. 27 illustrates a method of simulating motions in a virtual environment using a wearable article with flexible circuits, according to at least one non-limiting aspect of the present disclosure; and

FIG. 28 illustrates another article configured to track physical motions of a user, according to at least one non-limiting aspect of the present disclosure.

Corresponding reference characters indicate corresponding parts throughout the several views. The exemplifications set out herein illustrate various aspects of the invention in one form, and such exemplifications are not to be construed as limiting the scope of the invention in any manner.

### DETAILED DESCRIPTION

Numerous specific details are set forth to provide a thorough understanding of the overall structure, function, manufacture, and use of the aspects as described in the disclosure and illustrated in the accompanying drawings. Well-known operations, components, and elements have not been described in detail so as not to obscure the aspects described in the specification. The reader will understand that the aspects described and illustrated herein are non-limiting examples, and thus it can be appreciated that the specific structural and functional details disclosed herein may be representative and illustrative. Variations and changes thereto may be made without departing from the scope of the claims. Furthermore, it is to be understood that such terms as “forward,” “rearward,” “left,” “right,” “upwardly,” “downwardly,” and the like are words of convenience and are not to be construed as limiting terms.

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves any and all copyrights disclosed herein.

Electronic circuits that are flexible and deformable have emerged as a means of innovating conventional electronics and introducing electronics into new products and applications. However, it would be beneficial for flexible electronic circuits to form a sealed internal cavity, which can be filled with a compressible fluid. Such circuits could expand and contract in accordance with the selective insertion and/or removal of the fluid from the internal cavity. Moreover, the change in circuit geometry could lead to a subsequent change in electrical parameters generated across the inflatable circuit, which could be used to characterize a structural parameter or condition of the circuit as desired. Indeed, inflatable circuits could provide numerous benefits for airbags, bladders, and/or cushions, which could be calibrated, monitored, and even controlled based on measured electrical parameters. Accordingly, there is a need for devices, systems, and methods for making and using an inflatable circuit.

While certain electronic components typically have some inherent flexibility, that flexibility is typically constrained in the amount the components can flex, their resilience in flexing, and the number of times the electronic components can flex before the electronic components deteriorate or break. Consequently, the utility of such electronic components in various environments may be limited, either by reliability or longevity, or by the ability to function at all. Moreover, the lateral size of such components may result in additional stresses placed on the component.

The use of conductive gel, however, provides for electronic components that are flexible and deformable while maintaining resiliency. Moreover, the operational flexing, stretching, deforming, or other physical manipulation of a conductive trace formed from conductive gel may produce predictable, measurable changes in the electrical characteristics of the trace. By measuring the change in resistance or impedance of such a trace, the change in length of the trace may be inferred. By combining the changes in length of multiple traces, the relative movement of points on a two-dimensional surface may be calculated.

A two-dimensional strain sensor has been developed that utilizes a network of conductive gel traces, the individual electrical characteristics of which translate to a relative length or other orientation of the trace. By combining the electrical characteristics, e.g., by triangulating or by other mathematical process, the relative location of various points on a two-dimensional surface may be determined. By measuring such electrical characteristics repeatedly over time, the motion of the points may be determined, providing for the capacity for real-time motion capture of the points on the strain sensor. By scaling the network of traces and/or increasing the number of strain sensors and placing the strain sensors on an object, motion capture of the object may be obtained in real-time.

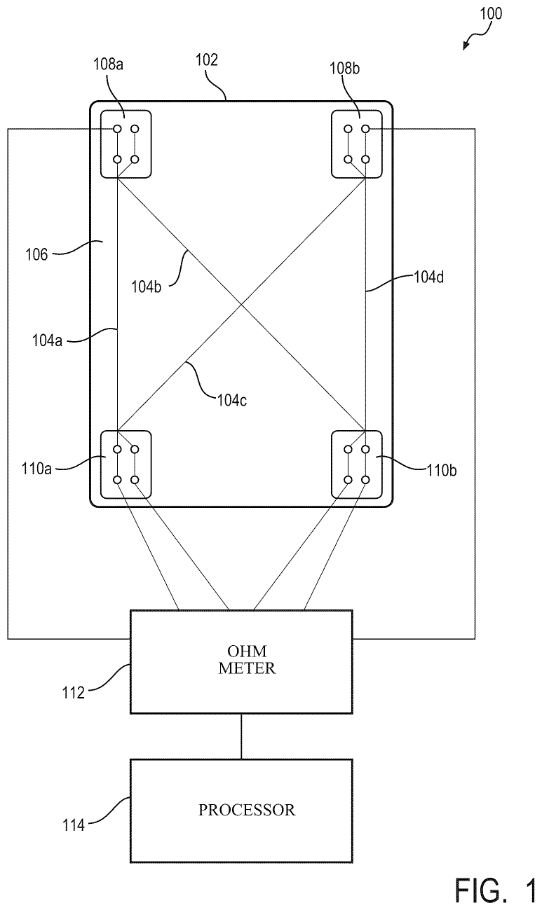

FIG. 1 is a view of a strain sensor system 100 including a two-dimensional strain sensor 102, in an example embodiment. As an example, the strain sensor system 100 can be configured similarly to those disclosed in U.S. Provisional Patent Application No. 63/263,112, titled TWO DIMENSIONAL MOTION CAPTURE STRAIN GAUGE SENSOR, filed Oct. 10, 2021, the disclosures of which are hereby incorporated by reference in its entirety. The strain sensor 102 includes four traces 104a, 104b, 104c, 104d. Each trace 104a-d is made of conductive gel, as disclosed in detail herein. The conductive gel is positioned on and encapsulated by a medium 106. Each trace 104a, 104b, 104c, 104d extends between and electrically couples one of two reference points 108a, 108b to an anchor point 110a, 110b. In the illustrated example, reference points 108a, 108b are not directly connected to one another and the anchor points 110a, 110b are not directly connected to one another.

The medium 106 specifically and the strain sensor 102 generally may be formed according to the techniques described herein or according to any other mechanism that exists or may be developed, including but not limited to injection molding, 3D printing, thermoforming, laser etching, die-cutting, and the like. The medium 106 may be formed of one of: a B-stage resin film, a C-stage resin film, an adhesive, a thermoset epoxy-based film, thermoplastic polyurethane (TPU), and/or silicone, among other suitable compounds or material. However, according to other non-limiting aspects, any materials may be used, assuming they can be unitized together. In an example, the medium 106 may comprise a layer that has a tensile elongation of 550%; tensile modulus of 5.0 megapascals; a recovery rate of 95%; a thickness of 100 micrometers; a peel strength at 90 degrees of at least 1.0 kilonewtons per meter; a dielectric constant of 2.3 at 10 gigahertz; a dielectric dissipation factor of 0.0030 at 10 gigahertz; a breakdown voltage of 7.0 kilovolts at a thickness of 80 micrometers; a heat resistance that produces no change in an environment of 260 degrees Celsius for 10 cycles in a nitrogen atmosphere; and a chemical resistance producing no change to the medium 106 after 24 hours immersion in any of NaOH, Na2CO3, or copper etchant.

Details of an example medium 106 are disclosed in U.S. Patent Application Publication No. 2020/0381349, “CONTINUOUS INTERCONNECTS BETWEEN HETEROGENEOUS MATERIALS,” Ronay et al., which is incorporated by reference herein in its entirety.

The strain sensor 102 is configured to identify changes in the relative positions of the reference points 108a, 108b based on a change in impedance/resistance of one or more of the traces 104a, 104b, 104c, 104d. In particular, the strain sensor 102 is configured to determine the relative position according to the Cartesian system (x,y) on a plane defined by the medium 106 of a given reference point 108a, 108b in relation to the two anchor points 110a, 110b, to which the reference point 108a, 108b is coupled via an associated trace 104a, 104b, 104c, 104d. Thus, for instance, the relative position of the reference point 108a may be determined by one or, inferentially, both of: determining the length at any given time of the trace 104a and the trace 104b and/or by determining the relative position (x,y) of the anchor points 110a, 110b.

The length of the traces 104a, 104b may be determined as a function of resistance and/or impedance of the given trace 104a, 104b, 104c, 104d as measured between the reference point 108a, 108b and the anchor point 110a, 110b that is coupled by the trace 104a, 104b, 104c, 104d. In the illustrated example, the strain sensor system 100 includes an electronic parameter sensor 112 operatively coupled to a processor 114. The electronic parameter sensor 112 may be any device that is configured to detect or otherwise measure an electronic property, such as resistance, capacitance, inductance, etc. As such, in various examples, the electronic parameter sensor 112 may be an ohm meter or a resistance signal reader. Further, the electronic parameter sensor 112 and the processor 114 may be separate components or integrated together. In such an example, the processor 114 may be part of a chipset or package that incorporates resistance signal reading and recording capabilities. In still yet other examples, an analog to digital signal processor may be utilized to convert an analog resistance signal to a digital signal, which may be received by the processor 114. In examples where a remote processor is configured to receive signals from the strain sensor 102, a wireless communication component integrated to the sensor may be configured to provide signals to the processor 114.

While the strain sensor system 100 as illustrated includes the electronic parameter sensor 112 and the processor 114, it is to be recognized and understood that one or both of the electronic parameter sensor 112 and the processor 114 may be remote to the rest of the strain sensor system 100 and/or cloud computing assets, etc. Moreover, in various examples the electronic parameter sensor 112 and/or the processor 114 may be integrated into the strain sensor 102 itself or may be components to which the strain sensor 102 is operatively coupled, as illustrated in FIG. 1. In examples where the processor 114 and/or the electronic parameter sensor 112 are remote to the strain sensor 102, a wireless communication module may be incorporated into the strain sensor 102 to provide data to the electronic parameter sensor 112 and/or processor 114.

In various examples, the processor 114 does not require a calibrated or predetermined relationship of impedance of a given trace 104a, 104b, 104c, 104d to determine the relative position of a reference point 108a, 108b and/or the relative position of an anchor point 110a, 110b. In such an example, the processor 114 may determine the relative location (x,y) on the medium 106 of the reference point 108a by determining the location of the reference point 108a relative to the determined location (x,y) of each of the anchor points 110a, 110b to which the traces 104a, 104b are coupled. In such an example, the location variables x and y of the reference point 108a may be determined by the processor 114 according to the following equations:

$$\begin{matrix}X - Coordinate of the Reference Point & \\ x = \frac{ℓ}{\partial } ( {x}_{a} - {x}_{b} ) \pm \frac{h}{\partial } ( {y}_{a} - {y}_{b} ) + {x}_{b} & Equation 1\end{matrix}$$

$$\begin{matrix}Y - Coordinate of the Reference Point & \\ y = \frac{ℓ}{\partial } ( {y}_{a} - {y}_{b} ) {-}_{+} \frac{h}{\partial } ( {x}_{a} - {x}_{b} ) + {y}_{b} & Equation 2\end{matrix}$$

$$h = \sqrt{{r}_{b}^{2} - {ℓ}^{2}} \qquad Equation 3$$

$$ℓ = \frac{{r}_{b}^{2} - {r}_{a}^{2} + {\partial }^{2}}{2 \partial } \qquad Equation 4$$

$$\partial = \sqrt{{( {x}_{a} - {x}_{b} )}^{2} + {( {y}_{a} - {y}_{b} )}^{2}} \qquad Equation 5$$

In the above equations, r is the impedance for a given trace 104a, 104b as measured by the electronic parameter sensor 112 and provided to the processor 114. By applying the same equations in the same manner for the reference point 108b, but for the traces 104c, 104d, the position of each of the reference points 108a, 108b may be determined. By performing the calculations at a relatively high frequency, e.g., at least once per second, or at least fifteen (15) times per second, or at least twenty-four (24) times per second, etc. The strain sensor system 100 may obtain a real-time determination of the relative positions of the reference points 108a, 108b and, therefore, the amount and rate of movement of the reference points 108a, 108b.

While the strain sensor system 100 is described with respect to the measurement of resistance or impedance, it is to be recognized and understood that any electrical measurement may be applied on a similar basis. Thus, for instance, the traces 104a, 104b, 104c, 104d may have or may be configured to have an inductance, a capacitance, or other measureable electronic property that may be changed based on a deformation of the trace. Consequently, while an electronic parameter sensor 112 is described and illustrated, it is to be recognized and understood that any electronic meter configured to sense and measure the relevant electronic property may be utilized in addition to or instead of the electronic parameter sensor 112 in a manner consistent with this disclosure.

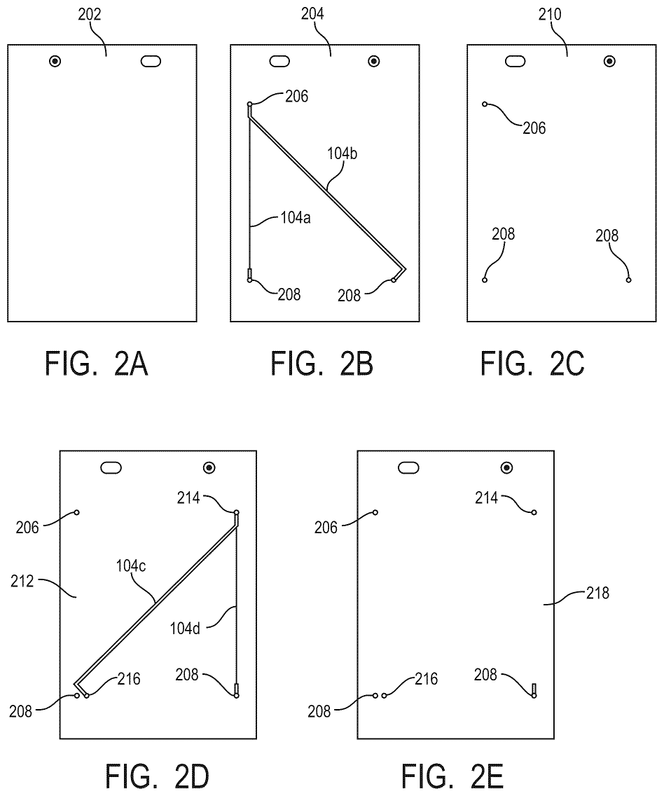

FIGS. 2A-2E are depictions of individual layers of the medium 106 of the strain sensor 102, in an example embodiment. In the example of FIGS. 2A-2E, the strain sensor 102 is a laminate structure in that individual layers of the medium 106 are separately formed, stacked, and unitized together to create the medium 106 as a whole. The layers may be formed according to iterative stencil-in-place processes described in U.S. Patent Application Publication No. 2020/0066628, titled “STRUCTURES WITH DEFORMABLE CONDUCTORS,” filed Aug. 22, 2019, the disclosure of which is hereby incorporated by reference in its entirety. However, as noted above, the formation of the strain sensor 102 as a laminate structure is for example and not to be construed as limiting, and any suitable technique for making the strain sensor 102 may be applied instead of or in addition to the process of making the strain sensor 102 as a laminate structure. The depictions of the layers are looking along a major axis of the strain sensor 102 and are thus either a top or bottom view of the layer relative to the perspective of FIG. 1.

According to some non-limiting aspects, the strain sensor 102 of FIGS. 2A-2E can be formed using any of the methods described in International Patent Application No. PCT/US2022/070850, titled DEVICES, SYSTEMS, AND METHODS FOR MAKING AND USING HIGHLY SUSTAINABLE CIRCUITS, and filed Feb. 25, 2022, the disclosure of which is herein incorporated by reference in its entirety. For example, according to some non-limiting aspects, a stencil layer can be melted and removed from the assembly after the deformable conductor is deposited onto a substrate layer and/or the deformable conductor can be reclaimed.

FIG. 2A depicts a substrate layer 202. The substrate layer 202 can be formed of one of the materials described above for the medium 106 and eventually has traces 104a, 104b placed thereon but is otherwise featureless and may, in various examples, provide insulation for and/or containment of the conductive gel.

FIG. 2B depicts a first patterned layer 204. The first patterned layer 204 is formed of another of the materials described above for the medium 106 and includes the traces 104a, 104b, which can be formed as channels that contain conductive gel formed in the medium 106. Additionally, a first reference via 206 and first anchor vias 208 are operatively coupled to the respective traces 104a, 104b and provide electrical access to the traces 104a, 104b through various layers of the strain sensor 102. The vias 206, 208 may be formed from conductive gel or any suitable conductor. Optionally, another patterned layer may be formed to include various features of the trace pattern such that first patterned layer 204 is a composite layer made from two individual layers.

FIG. 2C depicts an insulation layer 210. The insulation layer 210 is formed of another of the materials described above for the medium 106 and includes the first reference via 206 and the first anchor vias 208, which extend through the insulation layer 210.

FIG. 2D depicts a second patterned layer 212. The second patterned layer 212 is formed of another of the materials described above for the medium 106 and includes the traces 104c, 104d, which can be formed as channels that contain conductive gel formed in the medium 106. The first reference via 206 and the first anchor vias 208 extend through the second patterned layer 212, and a second reference via 214 and second anchor via 216 are operatively coupled to traces 104c, 104d. Optionally, another patterned layer may be formed to include various features of the trace pattern such that the second patterned layer 212 is a composite layer made from two individual layers.

FIG. 2E depicts an encapsulation layer 218. The encapsulation layer 218 is formed of another of the materials described above for the medium 106 and includes the first reference via 206, the first anchor vias 208, the second reference via 214, and the second anchor vias 216, all of which are exposed beyond the medium 106 to enable the strain sensor 102 to be operatively coupled to the electronic parameter sensor 112, as shown in FIG. 1.

The various layers are presented for illustration and not limitation, and it is to be recognized and understood that any of a variety of additional or alternative layers may be incorporated into the laminate structure as desired. The laminate structure may incorporate at least one substrate layer onto which conductive gel is positioned, at least one patterned layer that forms at least one trace, and at least one encapsulation layer that seals the trace or other component of the laminate structure. The laminate structure may further include: a stencil layer, e.g., for when a stencil-in-place manufacturing process is utilized; a conductive layer for, e.g., a relatively high-powered bus, sensor, ground plane, shielding, etc.; an insulation layer, e.g., between a substrate layer, a conductive layer, a stencil layer, and/or an encapsulation layer that primarily insulates traces or conductive layers from one another; an electronic component not necessarily formed according to the processes disclosed herein, e.g., a surface mount capacitor, resistor, processor, etc.; vias for connectivity between layers; and contact pads. The various layers can all be the same material, or one or more of the layers can be made from a different layer from the others, to form the laminate circuit structure.

The collection of layers of the laminate structure may be referred to as a “stack”. A final or intermediate structure may include at least one stack (or multiple stacks, e.g., using modular construction techniques) that has been unitized. Unitizing may involve one or more steps including the application of heat and/or pressure (including vacuum), and/or a curing operation, either alone or in combination. Additionally or alternatively, the structure can include one or more unitized stacks with at least one electronic component. A laminate assembly can include multiple laminate structures, for example in a modular construction. The assembly may utilize island architecture including a first laminate structure (the “island”), which may typically but not exclusively be itself a laminate structure populated with electric components, or a laminate structure that is, e.g., a discrete sensor, with the first laminate structure adhered to a second laminate structure including, e.g., traces and vias configured like a traditional printed circuit board (“PCB”), e.g., acting as the pathways for signals, currents or potentials to travel between the island(s) and other auxiliary structures, e.g., sensors.

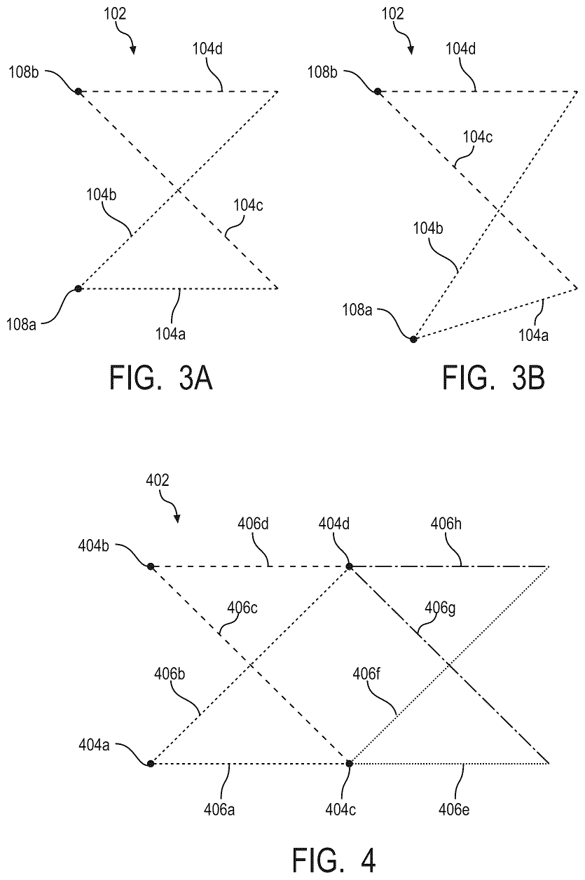

FIGS. 3A and 3B are abstract depictions of the traces of the strain sensor 102 in a relaxed and deformed configuration, respectively. The strain sensor 102 is considered to be in the relaxed configuration when an outside force is not acting on the strain sensor 102 such that the strain sensor 102 deforms through stretching, flexing, etc. The strain sensor 102 is considered to be in the deformed configuration when an outside force is acting on the strain sensor 102 such that the strain sensor 102 deforms through stretching, flexing, etc., and, as a result, one or more of the traces 104a, 104b, 104c, 104d lengthen or contract relative to their length in the relaxed configuration. It is noted that FIGS. 3A and 3B are described in a two-dimensional plane, but it is to be recognized and understood that the principles described with respect to two dimensions apply as well to three dimensional strain placed on the strain sensor 102.

In the illustrated example, in the relaxed configuration the traces 104a, 104d are of substantially equal length, e.g., within five (5) percent, and, as a result, of approximately equal resistance or impedance. Similarly, the traces 104b, 104c are similarly of substantially equal length and, as a result, of approximately equal distance. In such a circumstance, the processor 114 would determine that the relative (x, y) location of the reference points 108a, 108b are in their relaxed state.

In the deformed configuration, an outside force causes the reference point 108a to move relative to the reference point 108b. In the illustrated example, the length and, consequently, resistance of the traces 104c, 104d have not substantially changed, resulting in the processor 114 being configured to determine that, at least on a relative basis, strain has not been placed on the strain sensor 102 proximate the reference point 108b. However, the length and, consequently, the resistance of the traces 104a, 104b have changed, in the case of trace 104a to shorten, and in the case of trace 104b to lengthen relative to the length of those traces 104a, 104b in the relaxed state. Consequently, the processor 114 would be configured to determine that a strain has been placed on the strain sensor 102 proximate the reference point 108a.

Strain placed on the strain sensor 102 at different locations would result in different deformations of the strain sensor 102 and, consequently, different lengthening or shortening of the traces 104a, 104b, 104c, 104d than illustrated here. Moreover, while the length of two traces is shown as being constant, any or all of the traces 104a, 104b, 104c, 104d may change length and, consequently, measured resistance. Moreover, the strain sensor 102 may be sensitive to multiple forces placed on the strain sensor 102 to the extent that those different forces manifest at different locations on the strain sensor 102.

FIG. 4 is an abstract depiction of a strain sensor 402, in an example embodiment. In contrast to the strain sensor 102, the strain sensor 402 includes four reference points 404a, 404b, 404c, 404d. In such an example, the reference points 404c, 404d may function as de facto anchor points in relation to the reference points 404a, 404b. Consequently, the resistance over a trace 406a may be measured from reference point 404a to reference point 404c, and so forth.

The relative position of each reference point 404a, 404b, 404c, 404d are each determined by two of the traces 406. For the sake of clarity, the traces 406 associated with each reference point 404a, 404b, 404c, 404d are denoted by a particular dashed line. Thus, the relative position (x,y) of the reference point 404a is determined based on the resistance of the traces 406a, 406b, the relative position of the reference point 404c is based on the resistance of the traces 406e, 406f, and so forth. The principles disclosed herein are readily expandable to any number of reference points over any given area. The number of inputs on the electronic parameter sensor 112 or ohm meters may be expanded proportionally along with the processing resources of the processor 114.

Moreover, it is to be recognized and understood that the number of traces associated with a given reference point may expand based on the available traces. In various examples, the relative position of a reference point may be determined based on three or more traces rather than only two, with the equations described above expanded to incorporate the additional traces. However, in further examples the additional traces beyond two for each reference point 404 may be treated as redundant traces. Thus, the processor 114 may only utilize two traces to determine the relative position of a given reference point, but if a trace to a reference point 404 breaks then the processor 114 may utilize a different, unbroken trace to determine the relative position of the reference point 404.

The inclusion of multiple reference points 404 in a strain sensor and/or multiple strain sensors may provide for the creation of a real-time three dimensional model of a larger object. Thus, for instance, a wearable article may have traces extending throughout the wearable article, with the traces coupled to many reference points distributed throughout the wearable article. By regularly determining the relative position of each reference point, the processor 114 may readily create a three-dimensional model of the wearable article based on the change in relative position of each reference point to neighboring reference points.

Adaptation of the strain sensors disclosed herein to various use cases may result in the length of traces being optimized for the conditions of the wearable article or other article to which the strain sensor is attached. Thus, for instance, some traces may be relatively longer and the reference points spaced apart in certain locations that would not be expected to have strain placed thereon, e.g., along the fingers of a glove, while other traces may be relatively shorter and reference points spaced closer together in locations that may be expected to have strain placed thereon, for example, on a palm or the back of a hand of the glove.

The electrically conductive compositions, such as conductive gels, included in the articles described herein can, for example, have a paste like or gel consistency that can be created by taking advantage of, among other things, the structure that gallium oxide can impart on the compositions when gallium oxide is mixed into a eutectic gallium alloy. When mixed into a eutectic gallium alloy, gallium oxide can form micro or nanostructures that are further described herein, which are capable of altering the bulk material properties of the eutectic gallium alloy.

As used herein, the term “eutectic” generally refers to a mixture of two or more phases of a composition that has the lowest melting point, and where the phases simultaneously crystallize from molten solution at this temperature. The ratio of phases to obtain a eutectic is identified by the eutectic point on a phase diagram. One of the features of eutectic alloys is their sharp melting point.

According to some non-limiting aspects, the strain sensor 102 of FIGS. 2A-2E can be formed using any of the methods described in International Patent Application No. PCT/US2022/070853, titled DEVICES, SYSTEMS, AND METHODS FOR MAKING AND USING CIRCUIT ASSEMBLIES HAVING PATTERNS OF DEFORMABLE CONDUCTIVE MATERIAL FORMED THEREIN, and filed Feb. 25, 2022, the disclosure of which is herein incorporated by reference in its entirety. For example, according to some non-limiting aspects, the stencil layer can be omitted, as traces made from deformable conductors can be deposited directly on a substrate layer 202, as described in reference to FIG. 2A, and subsequently encapsulated without including a stencil layer in the final layup assembly. For example, the properties of the deformable conductive material and/or the properties of the layers surrounding the patterns of the deformable conductive material may be adjusted and/or optimized to ensure that the patterns of deformable conductive material heal upon unitization of the surrounding layers. For example, the deformable conductive material may be optimized to have a viscosity such that the deformable conductive material is able to heal upon unitization of the layers but not such that the deformable conductive material overly deforms and does not achieve the intended pattern. As another example, adhesive characteristics and/or viscosity of the deformable conductive material may be optimized such that it remains on the substrate layer upon removal of the removable stencil 50 but does not adhere to the channels 504, 506 of the stencil, thereby lifting the deformable conductive material off of the substrate layer. In some aspects, a viscosity of the deformable conductive material may, when under high shear (e.g., in motion), be in a range of about 10 Pascal seconds (Pa*s) and 500 Pa*s, such as a range of 50 Pa*s and 300 Pa*s, and/or may be about 50 Pa*s, about 60 Pa*s, about 70 Pa*s, about 80 Pa*s, about 90 Pa*s, about 100 Pa*s, about 110 Pa*s, about 120 Pa*s, about 130 Pa*s, about 140 Pa*s, about 150 Pa*s, about 160 Pa*s, about 170 Pa*s, about 180 Pa*s, about 190 Pa*s, or about 200 Pa*s. In some aspects, a viscosity of the deformable conductive material may, when under low shear (e.g., at rest), be in a range of 1,000,000 Pa*s and 40,000,000 Pa*s and/or may be about 10,000,000 Pa*s, about 20,000,000 Pa*s, about 30,000,000 Pa*s, or about 40,000,000 Pa*s.

The electrically conductive compositions described herein can have any suitable conductivity, such as a conductivity of from about 2×10'S/m to about 8×10'S/m.

The electrically conductive compositions described herein can have any suitable melting point, such as a melting point of from about −20° C. to about 10° C., about −10° C. to about 5° C., about −5° C. to about 5° C. or about −5° C. to about 0° C.

The electrically conductive compositions can include a mixture of a eutectic gallium alloy and gallium oxide, wherein the mixture of eutectic gallium alloy and gallium oxide has a weight percentage (wt %) of between about 59.9% and about 99.9% eutectic gallium alloy, such as between about 67% and about 90%, and a wt % of between about 0.1% and about 2.0% gallium oxide such as between about 0.2 and about 1%. For example, the electrically conductive compositions can have about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater, such as about 99.9% eutectic gallium alloy, and about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, and about 2.0% gallium oxide.

The eutectic gallium alloy can include gallium-indium or gallium-indium-tin in any ratio of elements. For example, a eutectic gallium alloy includes gallium and indium. The electrically conductive compositions can have any suitable percentage of gallium by weight in the gallium-indium alloy that is between about 40% and about 95%, such as about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

The electrically conductive compositions can have a percentage of indium by weight in the gallium-indium alloy that is between about 5% and about 60%, such as about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

The eutectic gallium alloy can include gallium and tin. For example, the electrically conductive compositions can have a percentage of tin by weight in the alloy that is between about 0.001% and about 50%, such as about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%.

The electrically conductive compositions can include one or more micro-particles or sub-micron scale particles blended with the eutectic gallium alloy and gallium oxide. The particles can be suspended, either coated in eutectic gallium alloy or gallium, and encapsulated in gallium oxide or not coated in the previous manner, within the eutectic gallium alloy. The micro- or sub-micron scale particles can range in size from nanometer to micrometer and can be suspended in gallium, gallium-indium alloy, or gallium-indium-tin alloy. Particle to alloy ratio can vary and can change the flow properties of the electrically conductive compositions. The micro and nanostructures can be blended within the electrically conductive compositions through sonication or other suitable means. The electrically conductive compositions can include a colloidal suspension of micro and nanostructures within the eutectic gallium alloy/gallium oxide mixture.

The electrically conductive compositions can further include one or more micro-particles or sub-micron scale particles dispersed within the compositions. This can be achieved in any suitable way, including by suspending particles either coated in eutectic gallium alloy or gallium and encapsulated in gallium oxide, or not coated in the previous manner within the electrically conductive compositions or, specifically, within the eutectic gallium alloy fluid. These particles can range in size from nanometer to micrometer and can be suspended in gallium, gallium-indium alloy, or gallium-indium-tin alloy. Particle to alloy ratio can vary in order to, among other things, change fluid properties of at least one of the alloys and the electrically conductive compositions. In addition, the addition of any ancillary material to colloidal suspension or eutectic gallium alloy can, among other things, enhance or modify its physical, electrical or thermal properties. The distribution of micro and nanostructures within the at least one of the eutectic gallium alloy and the electrically conductive compositions can be achieved through any suitable means, including sonication or other mechanical means without the addition of particles. In certain embodiments, the one or more micro-particles or sub-micron particles are blended with the at least one of the eutectic gallium alloy and the electrically conductive compositions with wt % of between about 0.001% and about 40.0% of micro-particles, for example about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40.

The one or more micro- or sub-micron particles can be made of any suitable material including soda glass, silica, borosilicate glass, quartz, oxidized copper, silver coated copper, non-oxidized copper, tungsten, super saturated tin granules, glass, graphite, silver coated copper, such as silver coated copper spheres, and silver coated copper flakes, copper flakes, or copper spheres, or a combination thereof, or any other material that can be wetted by the at least one of the eutectic gallium alloy and the electrically conductive compositions. The one or more micro-particles or sub-micron scale particles can have any suitable shape, including the shape of spheroids, rods, tubes, flakes, plates, cubes, prisms, pyramids, cages, and dendrimers. The one or more micro-particles or sub-micron scale particles can have any suitable size, including a size range of about 0.5 microns to about 60 microns, as about 0.5 microns, about 0.6 microns, about 0.7 microns, about 0.8 microns, about 0.9 microns, about 1 micron, about 1.5 microns, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, about 21 microns, about 22 microns, about 23 microns, about 24 microns, about 25 microns, about 26 microns, about 27 microns, about 28 microns, about 29 microns, about 30 microns, about 31 microns, about 32 microns, about 33 microns, about 34 microns, about 35 microns, about 36 microns, about 37 microns, about 38 microns, about 39 microns, about 40 microns, about 41 microns, about 42 microns, about 43 microns, about 44 microns, about 45 microns, about 46 microns, about 47 microns, about 48 microns, about 49 microns, about 50 microns, about 51 microns, about 52 microns, about 53 microns, about 54 microns, about 55 microns, about 56 microns, about 57 microns, about 58 microns, about 59 microns, or about 60 microns.

The electrically conductive compositions described herein can be made by any suitable method, including a method including blending surface oxides formed on a surface of a eutectic gallium alloy into the bulk of the eutectic gallium alloy by shear mixing of the surface oxide/alloy interface. Shear mixing of such compositions can induce a cross linked microstructure in the surface oxides; thereby forming a conducting shear thinning gel composition. A colloidal suspension of micro-structures can be formed within the eutectic gallium alloy/gallium oxide mixture, for example as, gallium oxide particles and/or sheets.

The surface oxides can be blended in any suitable ratio, such as at a ratio of between about 59.9% (by weight) and about 99.9% eutectic gallium alloy, to about 0.1% (by weight) and about 2.0% gallium oxide. For example, percentage by weight of gallium alloy blended with gallium oxide is about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater, such as about 99.9% eutectic gallium alloy while the weight percentage of gallium oxide is about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, and about 2.0% gallium oxide. In some embodiments, the eutectic gallium alloy can include gallium-indium or gallium-indium-tin in any ratio of the recited elements. For example, a eutectic gallium alloy can include gallium and indium.

The weight percentage of gallium in the gallium-indium alloy can be between about 40% and about 95%, such as about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

Alternatively or in addition, the weight percentage of indium in the gallium-indium alloy can be between about 5% and about 60%, such as about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

A eutectic gallium alloy can include gallium, indium, and tin. The weight percentage of tin in the gallium-indium-tin alloy can be between about 0.001% and about 50%, such as about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.4%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%.

The weight percentage of gallium in the gallium-indium-tin alloy can be between about 40% and about 95%, such as about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

Alternatively or in addition, the weight percentage of indium in the gallium-indium-tin alloy can be between about 5% and about 60%, such as about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

One or more micro-particles or sub-micron scale particles can be blended with the eutectic gallium alloy and gallium oxide. For example, the one or more micro-particles or sub-micron particles can be blended with the mixture with wt % of between about 0.001% and about 40.0% of micro-particles in the composition, for example about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%. In some embodiments the particles can be soda glass, silica, borosilicate glass, quartz, oxidized copper, silver coated copper, non-oxidized copper, tungsten, super saturated tin granules, glass, graphite, silver coated copper, such as silver coated copper spheres and silver coated copper flakes, copper flakes or copper spheres or a combination thereof, or any other material that can be wetted by gallium. In some embodiments the one or more micro-particles or sub-micron scale particles are in the shape of spheroids, rods, tubes, a flakes, plates, cubes, prisms, pyramids, cages, and dendrimers. In certain embodiments, the one or more micro-particles or sub-micron scale particles are in the size range of about 0.5 microns to about 60 microns, as about 0.5 microns, about 0.6 microns, about 0.7 microns, about 0.8 microns, about 0.9 microns, about 1 micron, about 1.5 microns, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, about 21 microns, about 22 microns, about 23 microns, about 24 microns, about 25 microns, about 26 microns, about 27 microns, about 28 microns, about 29 microns, about 30 microns, about 31 microns, about 32 microns, about 33 microns, about 34 microns, about 35 microns, about 36 microns, about 37 microns, about 38 microns, about 39 microns, about 40 microns, about 41 microns, about 42 microns, about 43 microns, about 44 microns, about 45 microns, about 46 microns, about 47 microns, about 48 microns, about 49 microns, about 50 microns, about 51 microns, about 52 microns, about 53 microns, about 54 microns, about 55 microns, about 56 microns, about 57 microns, about 58 microns, about 59 microns, or about 60 microns.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non-exclusive “or,” unless specifically stated otherwise.

According to some non-limiting aspects, a glove composed of one or more tubular structures, such as a joint monitoring sleeve, is disclosed. The glove can utilize an array of sensors, control circuitry, at least one user input device, and at least one display device. The glove can be made from one or more joint monitoring sleeves configured to be worn about a user's forearm, wrist, palm, and/or fingers and, for example, can be similar to those disclosed in U.S. Provisional Patent Application No. 63/157,812, titled JOINT MONITORING SLEEVE, filed Mar. 7, 2021, the disclosure of which is hereby incorporated by reference in its entirety.

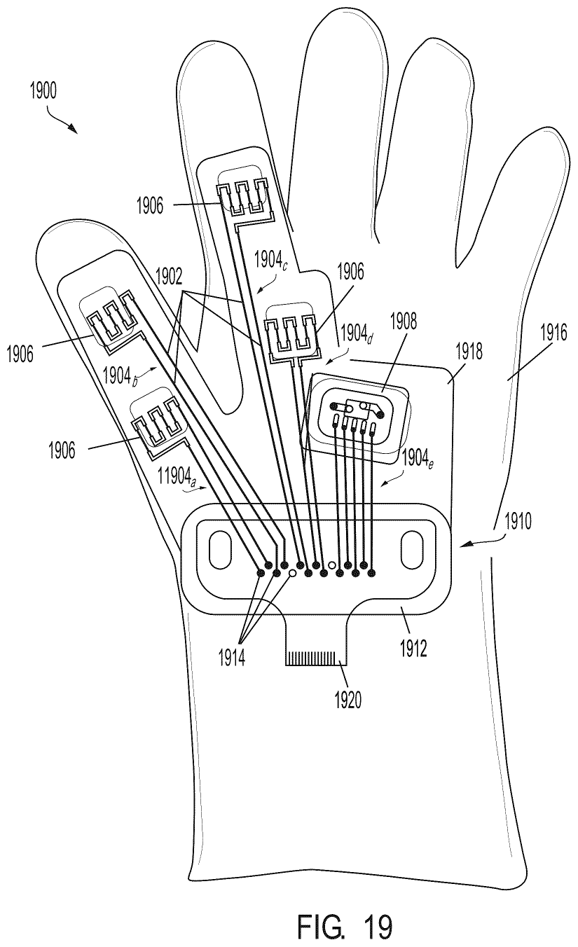

An embodiment of one such device contemplated by the present disclosure is shown beginning in FIG. 19, which implements several “sleeves” that are configured to be worn as a glove about a user's hand, fingers, and portions of the user's wrist and/or forearm. Alternatively, the sleeve may be configured to be worn as an elbow brace, an ankle brace, a wrist brace, or a brace for any anatomical joint that one may wish to monitor, implementing the principles described in reference to the illustrative glove of the present disclosure.

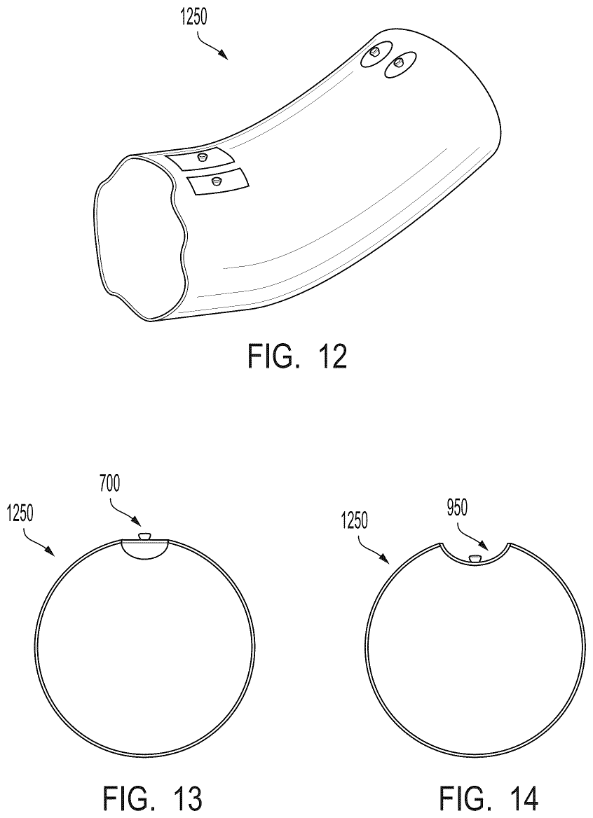

As will be described in further reference to FIGS. 12-14, the gloves disclosed herein can be formed from a substantially tubular member made from a textile, neoprene, or another material known for use as an athletic or medical brace. Since gloves of this type are intended to stretch in use, as well as when being positioned onto the body, they have typically been challenging to reliably instrument or to integrate electronics into without causing significant discomfort to the end user or experiencing high failure rates of integrated sensors and associated electronics.

The gloves described herein can utilize a deformable conductor and circuit manufacturing techniques disclosed in the above patent applications, which are incorporated by reference. Using these techniques and materials, it can be possible to seamlessly integrate an array of sensors onto the material of the glove in an unobtrusive fashion while producing reliable data on several parameters relating to the joint of the end user, for example, the user's hand.

Range of motion during hand flexion can be essential for tracking a user's motions while wearing a glove and can be a key indicator of hand joint health. Our instrumented glove actively monitors the hand joint flexibility of patients during activity. The strain sensor is enabled by a trace formed from a deformable conductor that moves with the joint. And with no degradation over thousands of strain cycles, there is no ongoing calibration needed. In addition, a smaller strain sensor may be placed near the front of the shin to measure swelling.

According to some non-limiting aspects of the present disclosure, several types of electromyogram (“EMG”) sensors commercially available can be used by the gloves described herein, many of which may be adequately functional for most wearers and glove sizes in combination. Examples of EMG sensors that may be used generally include dry and wet electrode style EMG sensors. It should be appreciated that the use of conductive gels for a wet electrode may typically provide the most reliable signal and may also be the least convenient and comfortable for a user over extended periods of use due at least to the associated messiness of a conductive gel. Therefore, in preferred embodiments a dry electrode EMG sensor is incorporated into the glove.

Examples of electrode types that may be integrated into the glove include a flexible dry silver nanowire type electrode embedded in PDMS, such as those described in U.S. patent application Ser. No. 15/127,455, filed on Apr. 7, 2015, which is hereby incorporated by reference in its entirety. Other electrode types may include silver-silver chloride pellet type EMG electrodes, for example those manufactured by J+J Engineering, including the models SE-13 and SE-12. A variety of other electrode configurations may be utilized effectively as well, and the foregoing examples are provided for illustrative purposes only.

As may be appreciated, the above example electrodes are different in configuration, but may be used to gather similar biometric data and signals when integrated to the sleeve embodiments contemplated herein. Another similarity between these electrodes is that they are available with a relatively large surface area for contacting a wearer's skin. For example, the SE-12 electrode has a circular contact area having a diameter of approximately 8 mm, and the SE-13 electrode is similar but larger with a corresponding diameter of approximately 17 mm. The electrode may be manufactured in a variety of sizes, and in the above reference application does not contemplate EMG sensor sizing. In embodiments of the present invention, it may be beneficial and/or preferable to have a surface contact area of at least about 20 mm² which may correspond to, for example, a circular contact area having a diameter of approximately 5 mm, or a rectangular contact area of approximately 4.5 mm in both width and length. More preferably, the contact area may have a surface area of about 130 mm². As with the above examples, this may correspond to an electrode having a diameter of about 13 mm or an equal length and width of about 11.5 mm. In another example, the EMG sensor or electrode may have surface area as large as 900 mm².

Similar to the preceding examples, this may correspond to a surface contact area having a diameter of approximately 34 mm, or a same length and width of about 30 mm. At times, depending on the muscle group for which activity is being measured or monitored, a larger contact area may be acceptable. In such cases, the contact area for the electrode or EMG sensor may be limited by the available area of the sleeve member, which in turn may be dictated by the remaining electronics and sensors integrated into the sleeve and considerations of pliability, flexibility, stretchability, or other similar factors in relation to the joint intended to be contained within the sleeve. It should be appreciated that other shapes and configurations may be selected and may therefore have differing characteristic dimensions but still meet the areal limits provided above.

A challenge associated with the above example EMG sensors may be achieving adequate signal from the sensor in some use cases and conditions. Due to the variety of limb sizes that may be contained within the glove or sleeve, varying pressures may result in variable contact quality between some wearers' skin and the sensor.

While the aforementioned exemplary electrode and sensor configurations may provide acceptable data and/or signals for monitoring the intended activity in a user's muscle or muscle groups, applicant has discovered a novel modification and improvement to such commercially available and/or experimental electrodes. Applicant has further discovered that enhanced reliability and improved signals may be obtained by incorporating an improved EMG electrode design. Since the glove is a tubular member exerting a radial pressure on the back side of the sensor electrode, there will be an associated deflection of the user's skin at the surface contact between the contact surface of the electrode and the user's body. Instances where there is a mismatch or less than optimal pairing between the selected glove or sleeve size and the wearer's body member size, a reliable contact interface between the sensor and the wearer's skin may not be achieved. This may be particularly problematic when the selected glove or sleeve size provides a preferred level of fit or comfort to the wearer, but suboptimal reliability or consistency in the interface between the wearer's skin and the electrode. This may be due to a variety of factors, some of which are related. For example, insufficient deflection of the user's skin may not produce adequate or reliable contact with the sensor, and/or the glove or sleeve may not produce sufficient radial force to enable adequate or reliable contact with the sensor.

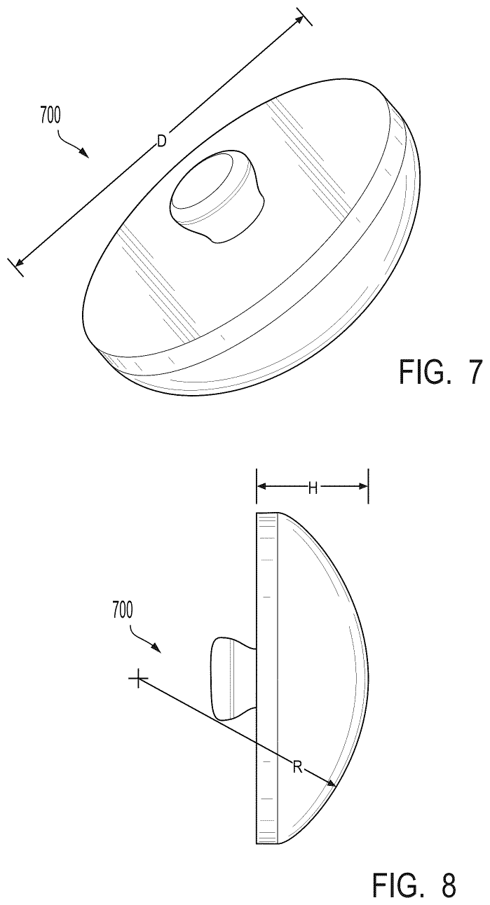

Examples of improved electrode configurations are shown in FIGS. 7-11, which introduce a convex radius to the contact surface. The electrodes, for example, can be configured similarly to any of the electrodes disclosed in U.S. International Patent Application No. PCT/US2022/071012, titled DEVICES, SYSTEMS, AND METHODS TO MONITOR AND CHARACTERIZE THE MOTIONS OF A USER VIA FLEXIBLE CIRCUITS, filed Mar. 7, 2022, the disclosure of which is hereby incorporated by reference in its entirety.

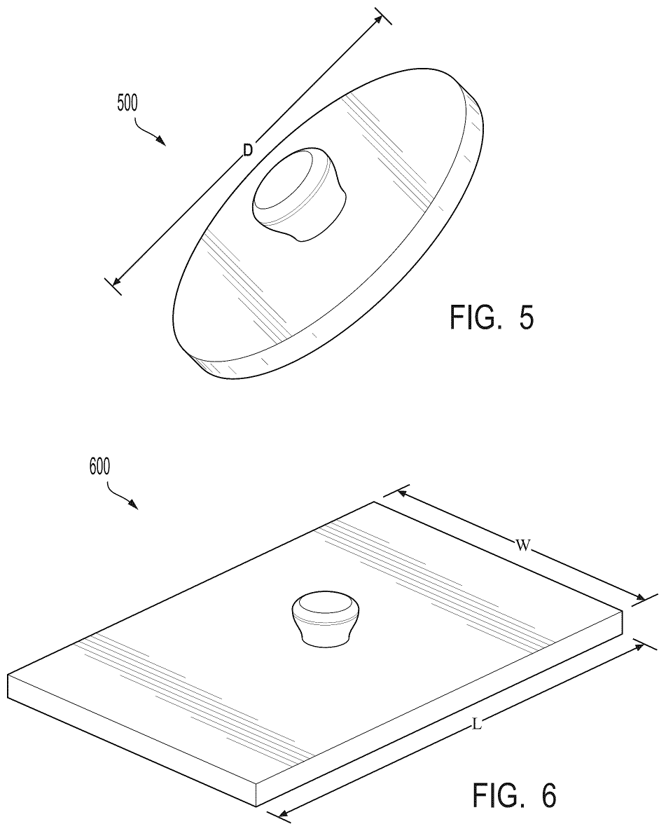

Referring now to FIGS. 5-11, several electrodes 500, 600, 700, 900, 950 that can be implemented via the wearable articles are disclosed herein, according to at least one non-limiting aspect of the present disclosure. Specifically, FIG. 5 depicts a round, flat electrode 500 defined by diameter D. According to FIG. 6, a rectangular, flat electrode 600 defined by width W and length L is depicted.

A “pellet” style electrode, or EMG, 700 is depicted in FIG. 7. The electrode 700 can, for example, be an electrode similar in general configuration to the round, flat electrode 500 of FIG. 5, including a similar diameter D. However, according to the non-limiting aspect of FIG. 7, the contact surface of the EMG 700 has been provided with a domed, spherical, or otherwise convex topography that approximates a radius R of about 0.5 to 1.5 times a major dimension of the electrode 700, such as diameter D. A round, pellet style electrode 700 similar to that of FIG. 7 can be provided with a major dimension, such as diameter D, of approximately 13 millimeters and a contact surface curvature radius R of approximately 11.5 millimeters, as depicted in FIG. 8. The electrode 700 of FIG. 7 can also include a spherical cap height H of 2 millimeters, as depicted in FIG. 8. However, other dimensions are contemplated by the present disclosure and can be implemented based on user preference and/or intended application. It should be appreciated that by introducing the domed shape to the electrode, the contact surface area is increased in comparison with a planar or flat electrode contact area, which may be beneficial In the above example, the resulting contact surface area is approximately 145 mm², whereas a flat contact surface electrode of the same outer diameter would have a surface area of only about 133 mm². Thus, it should be appreciated that an additional advantage of providing a curved contact surface is the ability to provide a larger contact area for a given form factor or “footprint” of any given electrode.

Furthermore, in comparison to a flat electrode integrated to a glove, as disclosed herein, the protrusion of the curved surface in relation to the surrounding glove surface may subtly concentrate the radial compressive forces of the glove on the wearer's skin at the electrode location, causing increased deflection and improved contact between the sensor and the wearer. One such electrode integrated into a sleeve, which can be used to form the gloves disclosed herein, is shown generally in FIGS. 12-14.

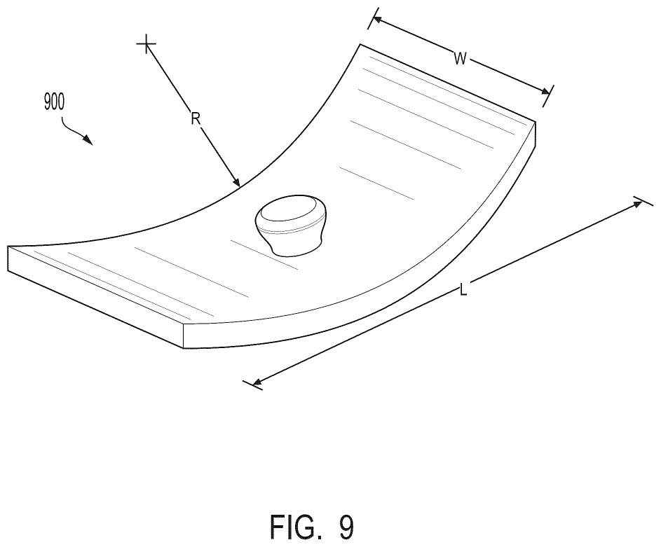

For a flat, sheet-style electrode 900 as shown in FIG. 9, the electrode is molded or otherwise formed to have a radius of curvature R that extends along substantially an entire length L or width W of the sheet. Either L or W may be considered a major dimension, which may be determined by the direction of radial axis. For example, in FIG. 9 the major dimension may be the length L since the radial axis extends in the width-wise direction, whereas if it were to extend in the lengthwise direction the major dimension may be taken to be the width W. The resulting structure behaves much like a leaf spring when integrated into a glove or sleeve, as depicted and described in reference to FIGS. 12-14. This configuration can provide a biasing force against the wearer's skin in response to the radial compression force supplied by the glove or sleeve as it is stretched over a portion of a wearer's body. Contact quality is thereby improved at the contact surface to skin interface, and therefore produces more reliable signals and/or data.

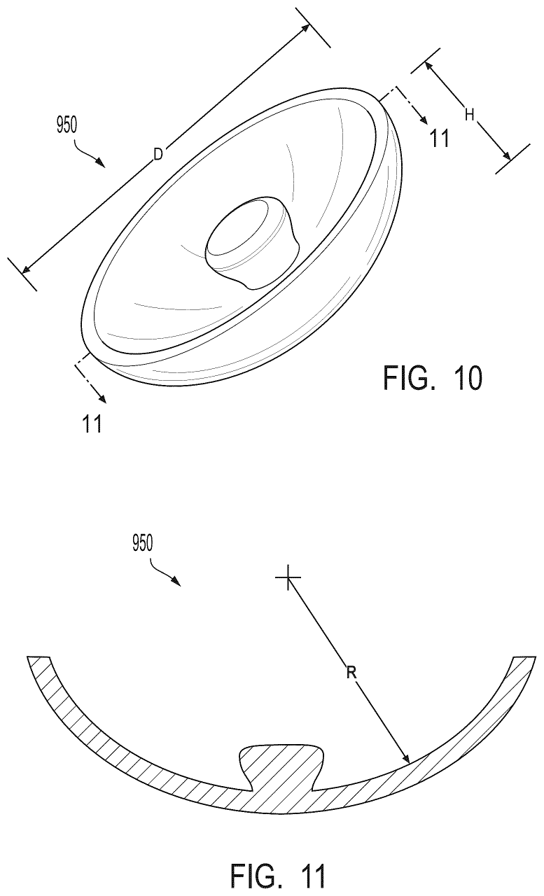

An alternative electrode 950 to the leaf spring style electrode 900 of FIG. 9 is depicted in FIGS. 10 and 11. According to the non-limiting aspect of FIGS. 10 and 11, an electrode 950 can have a cupped configuration in conjunction with a sheet-style electrode configuration. Similar to the electrode 900 of FIG. 9, when integrated into a glove or sleeve, the flexibility of the sheet electrode 950, in combination with the domed curvature, can produce a spring-like effect which biases the electrode 950 against the user's skin providing supplemental pressure and improved performance of the electrode. Although shown here with a generally circular shape, it should be appreciated that any shape of electrode may be provided with a domed or generally spherical topography. Here, the major dimension may be the diameter D of the electrode 950, as depicted in FIG. 10.

The electrodes 500, 600, 700, 900, 950 of FIGS. 5-11 may be molded or otherwise formed using an injection molding operation, casting operation, thermoforming operation or other suitable technique depending on the materials used to form the electrode and the desired characteristics or biasing effect necessary for a resulting sensor integration, for example, into a wearable apparatus, such as the glove or sleeve described herein and shown in FIGS. 12-14.

Referring now to FIGS. 12-14, a sleeve 1250 from which the wearable articles disclosed herein can be formed is depicted in accordance with at least one non-limiting aspect of the present disclosure. As previously described, the sleeve 1250 can be formed from a substantially tubular member made from a textile, neoprene, or another material known for use as an athletic or medical brace. Since gloves are intended to stretch in use and are worn proximal to a user's body, it has typically been a challenge to reliably instrument and/or integrate electronics into such articles without causing significant discomfort to the end user or experiencing high failure rates of integrated sensors and associated electronics. However, the non-limiting aspects of FIGS. 12-14 depict how various electrodes 700, 950 can be integrated into the sleeve 1250 and thus, a glove formed from the sleeve 1250, while preserving functional reliability and user comfort. Likewise, the flexible circuits and strain sensors described herein can be laminated or otherwise coupled to the sleeve 1250 to further promote functionality, flexibility, and user comfort, as will be described in further detail with reference to forthcoming figures.

Moreover, the sleeve 1250 of FIGS. 12-14 can further include an array of light emitting diodes (“LEDs”) that allows the patient or care provider to easily monitor flexion range in real time. This indicator can also be used to guide the patient through range of motion exercises during rehabilitation. An inductive coil sensor created using deformable conductors can be integrated into the glove to collect pressure information. Changes in output from this force sensor can be monitored as an indicator of swelling. Professionals will be able to clearly see pressure changes over a large area with the strain sensor and in a localized zone with the inductive coil. In addition to a force sensor, the sleeve 1250 can include an integrated temperature sensor built from deformable conductors. This can allow a user to monitor temperature changes in the injured area which can indicate a change in blood flow.

Electromyogram (EMG) readings can be used to diagnose conditions affecting muscles in the region. This output can be used during physical therapy or be used to control active prosthetics, among other uses. The EMG is a sophisticated active amplifier and filter that is created using a soft solder process in a highly pliable TPU film, such that contacts from the EMG or any other electronic component are placed in electrical communication prior to unitization. When the layers are unitized, the electrical connection is defined. Thus, it shall be appreciated that “soft soldering” a component can be particularly useful for modular assemblies or “stacks” of multiple layups, where various electrical connections must be defined and secured via unitization, which can be a function of curing in time, exposure to a ultraviolet light, etc. The sensor can pull voltages from skeletal muscle tissue using dry electrodes adhered directly to the TPU circuit, thereby resulting in a flexible, stretchable, fully conformable active circuit made from the deformable conductors described herein. This array of sensors can be integrated into the glove to be unperceivable to the end user.

Additionally, a deformable conductor can be used to make capacitive user input “buttons” of the sleeve 1250, which were integrated to the material of the sleeve 1250 and thus, the glove, such that touching the exterior surface of the glove in designated areas could cycle the functions of the glove to display different sensory outputs. Further, the capacitive input elements can be used to zero the feedback shown on the display or logged into memory for later retrieval. The buttons may be used by the end user to log a position in which the user feels discomfort, or an activity that results in pain, such as by adding a flag or tag to data being logged by onboard memory integrated into the control circuitry of the glove. Alternately, the buttons can be implemented to provide “touch” points for a virtual and/or augmented reality implementation of the gloves described herein.

Accordingly, it shall be appreciated that one or more sleeves similar to the sleeve 1250 of FIGS. 12-14 can be properly dimensioned and coupled together to form a glove that covers the user's hand, including the palm and one or more fingers, as well as portions of the user's wrist and/or forearm, depending on user preference and/or intended application.

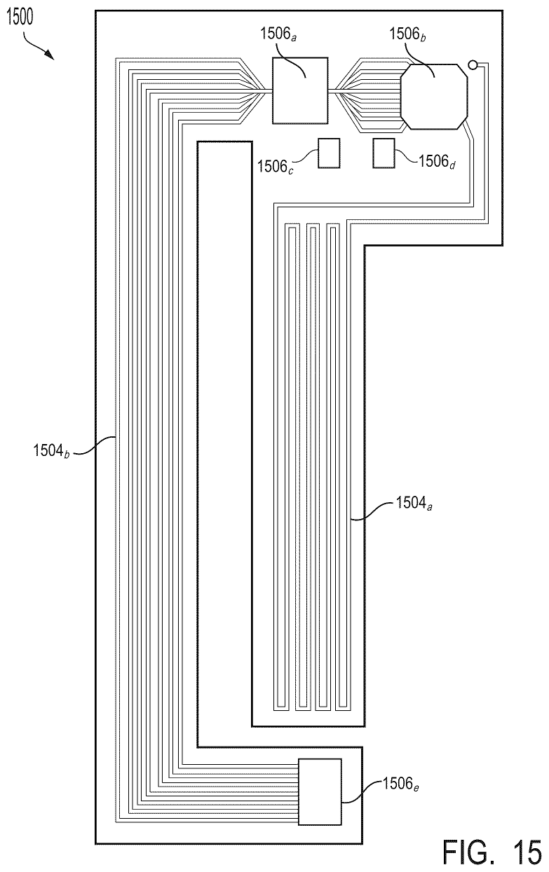

Referring now to FIG. 15, an alternative strain sensor 1500 that can be implemented to monitor a joint on a hand is depicted in accordance with at least one non-limiting aspect of the present disclosure. According to the non-limiting aspect of FIG. 15, the strain sensor 1500 of FIG. 15 can be configured to implementation on a wearable article, such as a glove, and arranged such that at least a portion of the strain sensor 1500 traverses at least a portion of a particular location of interest on the glove, for example, a finger of the glove. The strain sensor 1500 can implement the principles described in reference to the strain sensors 102, 402 of FIGS. 1-4, in that the strain sensor 1500 can be formed from traces 1504ₐ, 1504_{b} of deformable conductors that vary geometrically as the strain sensor 1500 is deformed. Specifically, the traces 1504ₐ, 1504_{b} can be formed from any of the deformable conductors described herein. However, the strain sensor 1500 of FIG. 15 is configured to not only monitor planar motion, but can be implemented on a wearable article to monitor a range of motions. For example, the elongated nature of the strain sensor 1500 can facilitate implementation along a finger of a glove and across one or more knuckles. Thus, as the strain sensor 1500 deforms, an algorithm or visualization engine stored in a memory of a computing device communicably coupled to the strain sensor 1500 can correlate the signals generated by one or more traces 1504ₐ, 1504_{b} of the circuit to a non-planar motion of the finger, or any other joint on the user's hand.

According to the non-limiting aspect of FIG. 15, the strain sensor 1500 can include one or more electronic components 1506ₐ₋ₑ, such as any of the processors, analog-to-digital converters (ADC), electrodes, memories, transceivers, power sources, inertial measurement units (“IMUs”), LED's, haptic sensors described herein, amongst other electronic components. Accordingly, a first trace 1504ₐ configuration composed of one or more traces of deformable conductors can be configured to measure positional displacement and second trace 1504_{b} composed of one or more traces of deformable conductors can be configured to function as a power and/or bus line for the strain sensor 1500, carrying data and/or electrical power between the various electronic components 1506ₐ₋ₑ and the first trace 1504ₐ of the strain sensor 1500. However, according to some non-limiting aspects, the second trace 1504_{b} can additionally and/or alternatively be configured to function as a strain sensor that monitors motions of the user's hand at a different location of interest on the user's hand.

The strain sensor 1500 of FIG. 15 can be integrated into one or more tubular sleeves, can be arranged along a length of the sleeve and can be oriented, generally, in an axial direction of the sleeve. For example, if the sleeve is configured to be worn over a user's hand, the strain sensor 1500 can be positioned to extend across one or more fingers of the user's hand. Generally, for applications over other joints, the sensor may generally be oriented transversely to an axis of articulation of the joint. In addition to the strain sensor, one or more of the electronic components 1506ₐ₋ₑ can include an IMU sensor, as will be described in further reference to FIG. 19. The IMU, for example, can be attached proximate each end of a sleeve, generally above and below a strain sensor. In other words, an IMU can be strategically positioned proximate the center of a limb, digit, or other body part intended to be contained within the sleeve. Using an IMU in conjunction with the strain sensor 1500 can, for example, improve joint monitoring.

Still referring to FIG. 15, the strain sensor 1500 can be positioned over a joint and may be used to correlate the measured strain (or stretch in the tubular sleeve) resulting from various relative angular relationships between limbs, digits, or other body members connected by a joint covered by the glove or any other wearable article. The measured strain may have a calibration for a plurality of angles and may infer the angles between the calibration points, for example, by assuming linear strain, which may be generally accurate for both metal gel conductor-based strain sensors and the bio-mechanics of the motion of body members covered by the wearable article, or glove. The addition of one or more electronic components 1506ₐ₋ₑ, such as IMUs, can add a symbiotic measure of an angle. One or more strain sensing traces 1504ₐ, 1504_{b} can calibrate and/or re-home data from an electronic component 1506ₐ₋ₑ, such as an IMU. The IMU can inform of motions that would act to add to the strain sensing traces 1504ₐ, 1504_{b}, like that of rotation at the joint or hyper extension beyond the set points of the strain sensor.

The use of two IMUs positioned on different positions opposite a joint (e.g., on either side of a knuckle, etc.) has been considered and can be implemented for inferencing joint movement and angular position of the fingers, but has been found to lack reliability over extended periods of use due to “drift” in the data provided by the IMUs. Over extended periods of time, the drift results in datasets that are not trustworthy, since the inferred position and spatial relationship between the IMUs is no longer within an acceptable tolerance of their actual position on the wearer's body. Attempting to understand limb and joint movements or rely on the data being provided by the IMU pair, for example, to remotely monitor the health of the joint or remotely perform physical therapy and training to rehabilitate the joint, is therefore not possible.

The addition of the strain sensor therefore provides not just data that is relatable to joint position and motion, but also serves to re-home the IMU's spatial position to generate more reliable data or extended periods of use. it may be necessary to benchmark associated strain and IMU-inferred spatial position data utilizing a calibration procedure for each wearer of a sleeve provided with this sensor configuration. This may be performed by the wearer moving their limb or body members contained in the sleeve to a variety of different positions and logging IMU inferred spatial location data versus measured strain. Thus, strain measurements may be used to anchor and correct the inferred spatial location of the IMUs as calculated by a Micro Controller Unit (“MCU”) integrated in some embodiments to the glove.

Typically, calibration of an IMU would not be possible with a strain sensor, since strain sensors are traditionally capable of measuring very small strains only, typically in the order of micrometers. Strains of such a small magnitude may be less than the drift in the spatial coordinates inferred by an IMU. A strain sensor made from the deformable conductors described herein may be capable of measuring strains in the order of centimeters and decimeters, and even greater magnitudes depending on the size of the sensor and the resilience of the substrate used to make the sensor. Thus, the use of a strain sensor to determine a correction factor to the drift in spatial position inferred by an IMU has considerable value to wearable electronics where translations of the IMUs as a result of relative motion of body parts results in substantial stretching of the wearable device by the user's body. Substantial stretching may be defined as linear stretch of 3 or more millimeters. In some applications, it may defined as little as about 1 millimeter. In other examples, it may be defined as 5 or 10 millimeters, or even more, depending on the use case of the sleeve.

The principles disclosed above may be applied to a sleeve fitted with a single IMU, which may provide substantially similar motion information for one finger, limb, digit, or other body member on either side of a joint of the wearer. The position of the other limb may be inferred from strain data.

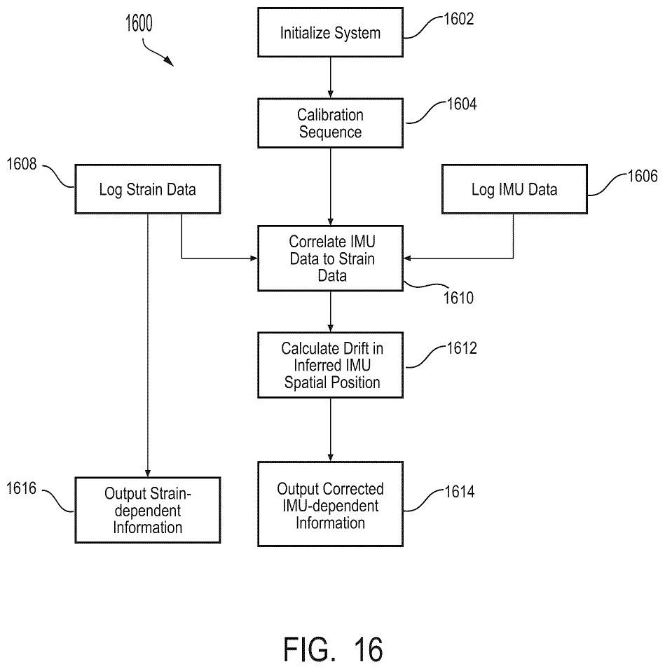

Referring now to FIG. 16, a method 1600 of correlating data generated by a strain gauge sensor to data generated by an inertial measurement unit (“IMU”) data is depicted according to at least one non-limiting aspect of the present disclosure. According to the non-limiting aspect of FIG. 16, the method 1600 can include initializing 1602 the system and then initializing 1604 a calibration sequence. The method 1600 can further call for logging 1608 strain data from one or more strain sensors on a glove and logging 1606 IMU data, from an IMU of a glove. The method 1600 can further include correlating 1610 IMU data to the strain data and calculating a drift 1612 in the inferred IMU spatial position based on the strain data. Finally, the method 1600 can include outputting 1616 strain-dependent information pertaining to the glove and outputting 1614 corrected IMU-dependent information.

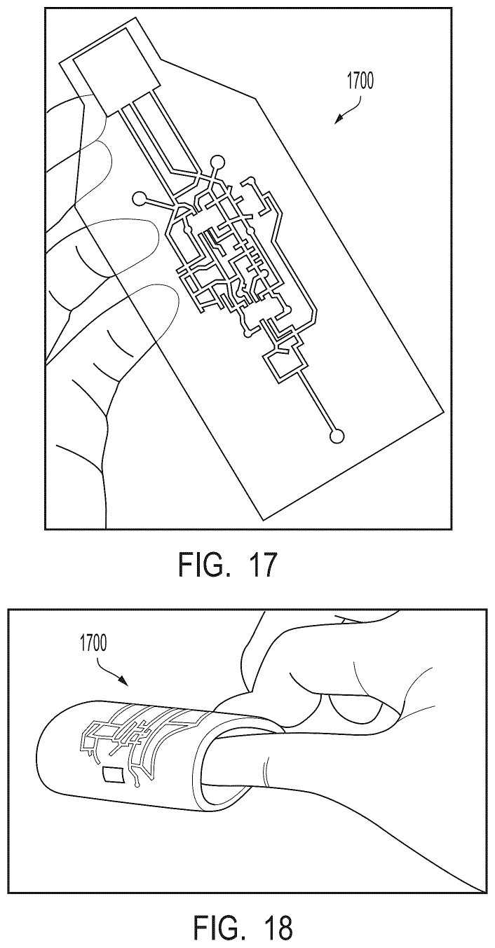

Referring now to FIGS. 17 and 18, another flexible circuit 1700 configured for use with the articles disclosed herein is depicted according to at least one non-limiting aspect of the present disclosure. For example, according to the non-limiting aspect, a flexible circuit 1700 that comprises traces made from deformable conductors, similar to the strain sensor 1500 of FIG. 15, is depicted in a relaxed condition. However, according to the non-limiting aspect of FIG. 18, the flexible circuit 1700 has been significantly deformed and is in a stressed condition. Accordingly, electrical parameters generated by the traces of deformable conductors will vary and the traces lengthen, due to the aforementioned nature of the deformable conductor. Thus, the flexible circuit 1700 of FIGS. 17 and 18 synthesizes the concepts described herein and is suitable for implementation via the gloves, systems, and methods for characterizing physical motions of a user, as described herein.

Referring now to FIG. 19, an article 1900 configured to track physical motions of a user is depicted in accordance with at least one non-limiting aspect of the present disclosure. For example, according to the non-limiting aspect of FIG. 19, the article 1900 can be configured as a glove to be worn on a user's hand. The glove 1900 can include certain elements that apply the aforementioned principles and techniques to generate electrical parameters, which can be correlated to physical parameters associated with a user's physical movements, when wearing the glove. Of course, according to other non-limiting aspects, the article can take the form of any other article of clothing, including a knee glove, a shirt, pants, a sock, and/or a hat, amongst others.

In further reference to FIG. 19, the glove 1900 can include a plurality of circuits 1904ₐ₋ₑ including one or more electrical features 1906, 1908, 1910 electrically coupled via a network of traces 1902 that are specifically configured to traverse various geometrical portions of the glove 1900. Any one of the glove 1900, the traces 1902, and/or the electrical features 1906, 1908, 1910 can be formed from a flexible and/or stretchable material. Accordingly, the glove 1900, the traces 1902, and/or the electrical features 1906, 1908, 1910 can enable the uninhibited motion of the user's hand while wearing the glove 1900, and can be used to generate electrical parameters that can be correlated to physical parameters associated with physical movements of the user, as will be described further herein. According to some non-limiting aspects, the traces 1902 can be deposited onto one or more substrates 1912, 1918, or layups, of the glove 1900 via the devices, systems, and methods disclosed in International Patent Application No. PCT/US2022/070853, titled DEVICES, SYSTEMS, AND METHODS FOR MAKING AND USING CIRCUIT ASSEMBLIES HAVING PATTERNS OF DEFORMABLE CONDUCTIVE MATERIAL FORMED THEREIN, and filed Feb. 25, 2022, and/or International Patent Application No. PCT/US2019/047731 titled STRUCTURES WITH DEFORMABLE CONDUCTORS, filed Aug. 22, 2019, the disclosures of which are hereby incorporated by reference in their entireties.

For example, the traces 1902 can utilize a flexible, deformable conductor, such as those disclosed in International Patent Application No. PCT/US2017/019762 titled LIQUID WIRE, which was filed on Feb. 27, 2017 and published on Sep. 8, 2017 as International Patent Publication No. WO2017/151523A1, the disclosure of which is hereby incorporated by reference in its entirety. For example, each trace 1902 can include a variety of forms, such as a liquid, a paste, a gel, and/or a powder, amongst others, that would enable the traces 1902 to have a deformable (e.g., soft, flexible, stretchable, bendable, elastic, flowable viscoelastic, Newtonian, non-Newtonian, etc.) quality. According to some non-limiting aspects, the deformable, conductive materials can include an electroactive material, such as deformable conductors produced from a conductive gel (e.g., a gallium indium alloy). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Of course, the present disclosure contemplates other non-limiting aspects, featuring traces 1902 of varying forms and/or compositions to achieve the benefits disclosed herein.

According to the non-limiting aspect of FIG. 19, the glove 1900 can include one or more substrates 1912, 1918 mounted to its primary material 1916, wherein the one or more substrates 1912, 1918 are composed of flexible and stretchable materials, such as those disclosed by U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. Specifically, the one or more substrates 1912, 1918 can be fabricated from a flexible or stretchable material such as a natural rubber, a synthetic rubber, a flexible plastic, a silicone based material (e.g., polydimethylsiloxane (“PDMS”), thermoplastic polyurethane (“TPU”), ethylene propylene dieneterpolymer (“EPDM”), neoprene, polyethylene terephthalate (“PET”), etc.), a flexible composite material, and/or a naturally flexible material, such as a leather, for example. For example, the one or more substrates 1912, 1918 can be fabricated from a resilient, albeit stretchable TPU, such as Lubrizol® Estane® 58000 series (e.g., 58238), amongst others. Alternatively, the one or more substrates 1912, 1918 can be formed from a flexible, though comparatively more rigid, material, such as Lubrizol® Estane® S375D, amongst others. According to other non-limiting aspects, the primary material 1916 of the glove 1900, itself, can include any of the aforementioned flexible and/or stretchable materials. Although the substrates 1912, 1918 of FIG. 19 can include a multi-layer construction—including a substrate layer, a stencil layer, and an encapsulation layer—in other non-limiting aspects, the substrates 1912, 1918 can include a two-layer construction (e.g., substrate layer, encapsulation layer, etc.) or even a single layer configured to accommodate the traces 1902.

Still referring to FIG. 19, the flexible and/or stretchable nature of the glove 1900, the traces 1902, and/or the electrical features 1906, 1908, 1910 can enable the generation of electrical parameters that can be correlated to physical parameters associated with physical movements of the user. For example, as the user dons the glove 1900 and moves their hand, the resulting physical disturbance to the traces 1902, and/or the electrical features 1906, 1908, 1910 mounted to the primary material 1916 can subsequently vary the electrical parameters (e.g., an inductance, a resistance, a voltage drop, a capacitance, and an electromagnetic field, etc.) generated by the traces 1902, and/or the electrical features 1906, 1908, 1910. In other words, the user's motions while wearing the glove 1900 can result in deformation of the traces 1902 and/or the electrical features 1906, 1908, 1910 which will alter electrical parameters that can be correlated to baseline data-which can be gathered using methods that will be discussed in further detail herein—to monitor and/or characterize the motion of the user's hand while wearing the glove 1900. The electrical parameters (e.g., an inductance, a resistance, a voltage drop, a capacitance, and an electromagnetic field, etc.) generated by the electrical features 1906, 1908, 1910 can be correlated to physical parameters (e.g., a strain, a stress, a pressure, a dimension, etc.) associated with the electrical features 1906, 1908, 1910 and, thus, can characterize the motion of the user's hand while wearing the glove 1900. The differences in correlated physical parameters of each circuit 1904ₐ₋ₑ can be used to model the user's hand in a virtual environment.

The electrical features 1906, 1908, 1910 of the glove 1900 can include a particular trace configuration 1906 and/or an IMU 1908, amongst other components specifically configured to generate signals that can be correlated to physical parameters of the glove 1900 (e.g., gyroscopes, accelerometers, magnetometers, pressure sensors, etc.). For example, according to other non-limiting aspects, a micro-electrical mechanical system (“WEMS”) gyroscope could also be employed. Specifically, each circuit 1904ₐ₋ₑ of the glove 1900 of FIG. 19 can include a particular trace configuration 1906 in one or more portions of the glove 1900.

According to the non-limiting aspect of FIG. 19, the particular trace configurations 1906 employed by the glove 1900 can include a series of “switch backs,” wherein the trace 1902 loops back on itself, thereby extending the length of the trace 1902 in that particular portion of the glove 1900. The portions of the glove 1900 wherein the particular trace configurations 1906 are positioned may be of specific interest to the user. For example, according to the non-limiting aspect of FIG. 19, the particular trace configurations 1906 can be positioned at approximately an estimated position of a user's knuckle when wearing the glove 1900. Specifically, a first circuit 1904ₐ and a fourth circuit 1904_{d} of the glove 1900 can include a particular trace configuration 1906 positioned about the approximate position of a user's most proximal knuckle. Likewise, a first circuit 1904ₐ and a fourth circuit 1904_{d} of the glove 1900 can include a particular trace configuration 1906 positioned about the approximate position of a user's intermediate knuckle. As such, each particular trace configuration 1906 can undergo an exacerbated deformation when the user moves their hand, resulting in more dramatic variations in electrical parameters and more accurate characterizations the motion of the user's hand while wearing the glove 1900. Of course, according to other non-limiting aspects, other geometric arrangements for the particular trace configurations 1906 are implemented. Each particular trace configuration 1906 need only have a different geometric arrangement than the rest of the traces 1902 of the circuit 1904ₐ₋ₑ.

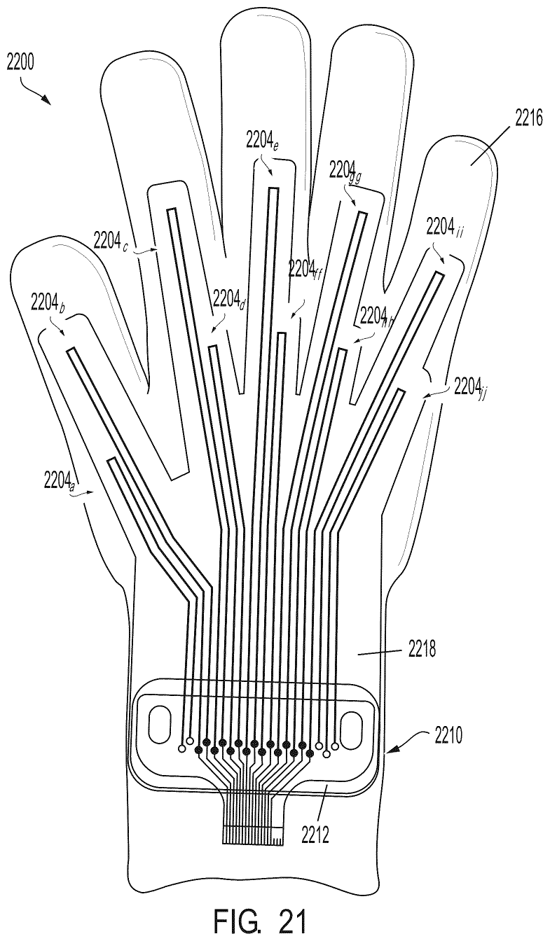

Although the glove 1900 of FIG. 19 depicts a first circuit 1904ₐ and a fourth circuit 1904_{d} positioned about the user's thumb and pointer finger, other non-limiting aspects can include various circuits 1904ₐ₋ₑ positioned about any finger or any other portion of the user's hand that may be of particular interest. Of course, according to other non-limiting aspects (e.g., the glove 2100 of FIG. 21), no particular trace configurations 1906 can be employed, and electrical parameters generated by the traces 1902 themselves can be correlated to physical parameters to characterize the motion of the user's hand while wearing the glove 1900.

Additionally and/or alternatively, one or more circuits 1904ₑ of the glove 1900 can include an IMU 1908 positioned in approximately the palm of the glove 1900, which can be configured to generate signals, which-according to some non-limiting aspects, in conjunction with signals generated by one or more other circuits 1904_{l-f}—can be correlated to physical parameters of the glove 1900 and used to characterize a user's motions when wearing the glove 1900. For example, it shall be appreciated that the IMU 1908 of FIG. 19 can include a number of accelerometers, which can output linear acceleration signals on three axes in space, and/or gyroscopes, which can output angular velocity signals on three axes in space, to measure triaxial acceleration and/or angular velocity of the user's hand while wearing the glove 1900. It shall be further appreciated how, in conjunction with the other circuits 1904_{a-d}, the IMU 1908 can be used to determine other aspects of a position and orientation (“POSE”) of the glove 1900 in three-dimensional space. For example, the various traces 1902 and particular trace configurations 1906 of the other circuits 1904_{a-d} can generate electrical parameters (e.g., an inductance, a resistance, a voltage drop, a capacitance, and an electromagnetic field, etc.), which can be used to contextualize and/or calibrate signals generated by the IMU 1908. Accordingly, if the IMU 1908 begins to drift due to extended use, a processor communicably coupled to the circuits 1904ₐ₋ₑ can utilize signals associated with electrical parameters from the other circuits 1904_{a-d} to correct signals received from the IMU 1908.

According to some non-limiting aspects, the IMU 1908 can include an onboard construction, including traces that are constructed of a deformable conductor, similar to the traces 1902 of the individual circuits 1904_{a-d}. As such, deformations within the IMU 1908 itself can be utilized to contextualize and/or calibrate signals generated by other components IMU 1908 (e.g., gyroscopes, accelerometers, magnetometers, pressure sensors, etc.). In other words, according to some non-limiting aspects, the IMU 1908 can be constructed according to U.S. Provisional Patent Application No. 63/261,266, titled STRETCHABLE AND FLEXIBLE METAL FILM STRUCTURES, filed Sep. 21, 2021, to reduce the need for additional circuits 1904_{a-d}.

In further reference to FIG. 19, the electrical features 1906, 1908, 1910 can further include a coupling circuit 1910 configured to couple the traces 1902 of the circuits 1904ₐ₋ₑ of the glove 1900 to a processing circuit via a plurality of vias 1914, such as those disclosed in U.S. Provisional Patent Application No. 63/261,266, titled STRETCHABLE AND FLEXIBLE METAL FILM STRUCTURES, filed Sep. 21, 2021, the disclosure of which is hereby incorporated by reference in its entirety. For example, the traces 1902, vias 1914, and contacts (not shown) may be particularly sized and spaced to establish the desired electrical connections such that signals generated by the circuits 1904ₐ₋ₑ of the glove 1900 can be transmitted to a processor via an electrical connector 1920. Of course, according to other non-limiting aspects, the coupling circuit 1910 can be hardwired to the processor. The processor can be communicably coupled to a memory configured to store instructions that, when executed by the processor, cause the processor to characterize the user's motion while wearing the glove 1900. The processor can be coupled to a display that can be configured to present a virtual representation of the glove—and thus, the user's movements—in a virtual environment. Alternately, the coupling circuit 1910 can be configured for conventional wireless (e.g., infrastructure networks, such as WiFi®, cellular, etc., and/or ad hoc networks, such as Bluetooth®, near-field communication (“NFC”), radio-frequency identification (“RFID”), etc.) transmissions. According to some non-limiting aspects, any of the electrical features disclosed herein, such as an IMU 1908, the coupling circuit 1910, and/or the electrical connector 1920 can include a PCB construction including a polyimide flexible board construction that can be bonded to the laminate structure. Electrical contacts (not shown) and/or traces (not shown) hosted on such electrical features 1908, 1910, 1920—such as sensors and/or chipsets of the IMU 1908 or other components of the coupling circuit 1910, and/or the electrical connector 1920 (e.g., Bluetooth® radio, USB connectors, etc.)—can be reflow soldered to the flexible PCB. For example, according to some non-limiting aspects, the PCBs can be constructed as described in U.S. patent application Ser. No. 16/885,854, titled CONTINUOUS INTERCONNECTS BETWEEN HETEROGENEOUS MATERIALS, and filed May 28, 2020, the disclosure of which is herein incorporated by reference in its entirety. Alternatively and/or additionally, various chips and/or sensors can be directly hosted on the laminate circuit structure itself.

According to some non-limiting aspects, the processor can be remotely located relative to the glove 1900. According to other non-limiting aspects, the coupling circuit 1910 of the glove 1900 can further include an on-board processor such that signals generated by the circuits 1904ₐ₋ₑ can be locally processed and the coupling circuit 1910 can couple the glove to the display. Alternately and/or additionally, the coupling circuit 1910—which, according to the non-limiting aspect of FIG. 19, is positioned approximately about a user's wrist—can include one or more sensors (e.g., gyroscopes, accelerometers, magnetometers, pressure sensors, etc.) configured to generate electrical parameters, which can be correlated to physical parameters of the coupling circuit 1910 to characterize motions of the user's wrist. In some non-limiting aspects, the coupling circuit 1910 can include a rechargeable power source (e.g., a lithium-ion battery, a capacitor, etc.) configured to deliver an electrical current to the circuits 1904_{e-f} and/or a port (e.g., a universal serial bus (“USB”) port) configured to directly deliver an electrical current to the circuits 1904_{e-f} and/or charge the power source itself.

Still referring to FIG. 19, one or more substrates 1912, 1918 or portions of the primary material 1916 can be fabricated from a more resilient, albeit stretchable TPU, such as Lubrizol® Estane® 58000 series (e.g., 58238), amongst others. Alternatively, the one or more substrates 1912, 1918 can be formed from a flexible, though comparatively more rigid, material, such as Lubrizol® Estane® S375D, amongst others. Accordingly, one or more substrates 1912, 1918 or portions of the primary material 1916 can be reinforced to limit and/or restrict deformations of certain traces 1902 and/or electrical features 1906, 1908, 1910 altogether, or in a particular axis, such that electrical parameters do not vary as much relative to other traces 1902 and/or electrical features 1906, 1908, 1910 of interest. In other words, the relative flexibility and rigidity of various portions and/or components of the glove 1900 can be used to ensure signals generated by the circuits 1904ₐ₋ₑ carry information relevant to areas of interest. This can lead to more efficient processing and thus, enhance the accuracy and economic value of characterizations generated by the glove 1900.

According to still other non-limiting aspects, the glove 1900 can include a variety of other electrical features, such as pressure sensors. According to one non-limiting aspect, the glove 1900 can include a pressure sensor on the tip of one or more fingers. The pressure sensor, for example, can include any of those described in International Patent Application No. PCT/US2021/071374, titled WEARABLE ARTICLE WITH FLEXIBLE INDUCTIVE PRESSURE SENSOR, filed Sep. 3, 2021, U.S. Provisional Application No. 63/270,589, titled FLEXIBLE THREE-DIMENSIONAL ELECTRONIC COMPONENT, filed Oct. 22, 2021, and U.S. Provisional Application No. 63/272,487, titled DEVICES, SYSTEMS, AND METHODS FOR MAKING AND USING A FLUID-FILLABLE CIRCUIT, filed Oct. 27, 2021, the disclosures of which are hereby incorporated by reference in their entireties. Accordingly, as an inductive coil in the sensor is depressed or extended, an electrical parameter (e.g., an electromagnetic inductance, etc.) generated by the sensor will vary and corresponding signals can be transmitted via the circuits 1904ₐ₋ₑ to the processor for characterization of stimulations external to the glove 1900 that are being detected by the pressure sensor. Of course, other pressure sensors (e.g., strain gauges, thin film pressure sensors, variable capacitance pressure sensors, etc.) can be implemented to achieve a similar effect.

According to some non-limiting aspects, it may be useful to pair the glove 1900 of FIG. 19 with a smartphone that may run a dedicated app to provide additional functionality such as the ability to record a voice memo, for example, when logging data regarding use of the glove 1900 in a virtual and/or augmented reality implementation or for a physiotherapeutic implementation. Further, the data generated by the glove 1900 may be streamed wirelessly to cloud storage or monitored in real time by an individual in a remote location, for example, for providing therapeutic instructions or advice, exercises, training, or diagnosis of an injury. To achieve wireless communications with a device configured to receive data from the sleeve, a processor of the glove 1900 may be provided with a wireless module such as a Bluetooth® radio and associated firmware for enabling wireless communication and data transfer. Further, as described elsewhere herein, the glove 1900 can further include an ADC coupled to the processor between the strain sensor 1904ₐ₋ₑ and the processor to convert analog signals to digital signals for interpretation by the processor. Similar to the sleeve 1250 of FIGS. 12-14, according to some non-limiting aspects, the glove 1900 of FIG. 19 can seamlessly integrate an array of sensors, electrodes, control circuitry, at least one user input device, and at least one display device. Furthermore, according to other non-limiting aspects, the glove 1900 can include an array of LEDs, haptic sensors, transducers, and/or a visual display configured to provide the user with feedback pertaining to their flexion range in real time. With regards to a therapeutic implementation of the gloves described herein, this indicator can also be used to guide the patient through range of motion exercises during rehabilitation. According to still other non-limiting aspects, the glove 1900 can include an inductive coil sensor created from deformable conductors can be integrated into the glove to collect pressure information. Changes in output from this force sensor can be monitored as an indicator of swelling or, alternately, can be utilized to assess whether a user is grasping an object in their hand while wearing the glove 1900. It shall be appreciated that pressure changes can be monitored over a large area with one such strain sensor and, according to some non-limiting aspects, can be monitored in a localized zone with the inductive coil. In addition to the force sensor, according to some non-limiting aspects, the glove 1900 of FIG. 19 can include an integrated temperature sensor configured from deformable conductors. Such sensors can enable a user to monitor a temperature change in the user's hand while wearing the gloves described herein.

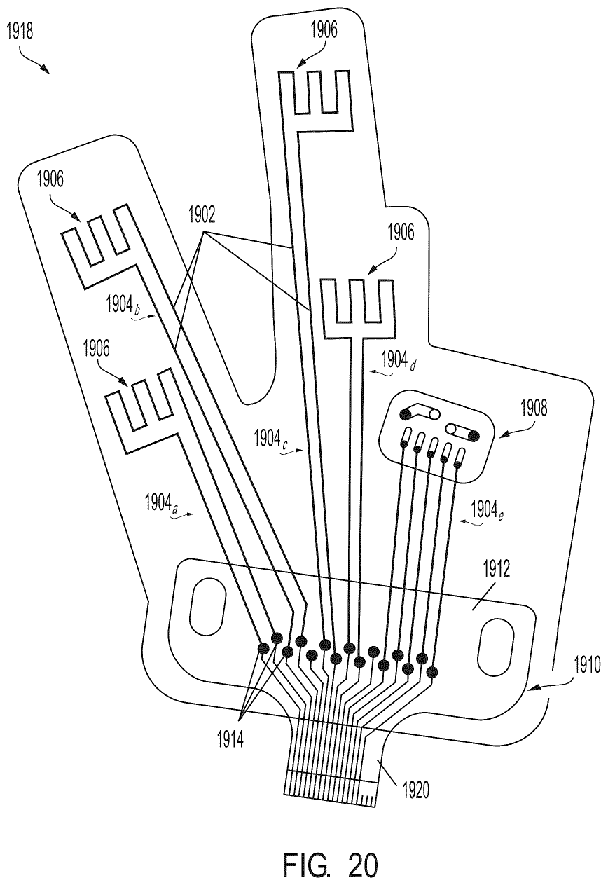

Referring now to FIG. 20, one of the substrates 1918 of the glove 1900 of FIG. 19 is depicted in accordance with at least one non-limiting aspect of the present disclosure. Specifically, FIG. 20 illustrates the modular construction of the glove 1900 of FIG. 19. According to the non-limiting aspect of FIG. 20, the circuits 1904ₐ₋ₑ, including the traces 1902 and the electrical features 1906, 1908, 1910, can be constructed as disclosed in International Patent Application No. PCT/US2022/070853, titled DEVICES, SYSTEMS, AND METHODS FOR MAKING AND USING CIRCUIT ASSEMBLIES HAVING PATTERNS OF DEFORMABLE CONDUCTIVE MATERIAL FORMED THEREIN, and filed Feb. 25, 2022, the disclosure of which is herein incorporated by reference in its entirety. After the substrate 1918 is constructed—with varying degrees of stretch and flexibility to promote and/or restrict deformations in the desired portions of the substrate 1918—the substrate 1918 can be mounted to the primary material 1916 of the glove 1900 of FIG. 19. The material of each substrate 1918, 1912 used during the construction can be particularly selected as well. For example, according to some non-limiting aspects, the substrate 1918 can be composed of the same material or have mechanical properties similar to those of the substrates 1912 that are mounted to it. For example, the substrates 1912, 1918 can have a similar elastic modulus or other elastic properties, which can reduce the chances of a shear mismatch between the substrates 1912, 1918, as the glove 1900 (FIG. 19) moves, which may result in delamination depending on the attachment, bonding, or coupling method selected to attach the components to one another.

Referring now to FIG. 21, another article 2200 configured to track the physical motions of a user is depicted in accordance with at least one non-limiting aspect of the present disclosure. Similar to the article 1900 of FIG. 19, the article 2200 can be configured as a glove to be worn on a user's hand. Once again, the glove 2200 can include certain elements that apply the aforementioned principles and techniques to generate electrical parameters, which can be correlated to physical parameters associated with a user's physical movements, when wearing the glove. Of course, according to other non-limiting aspects, the article can take the form of any other article of clothing, including a knee glove, a shirt, pants, a sock, and/or a hat, amongst others.

In further reference to FIG. 21, the glove 2200 can include a plurality of circuits 2204ₐ₋ₑ including a network of traces 2202 that are specifically configured to traverse various geometrical portions of the glove 2200. However, unlike the glove 1900 of FIG. 19, the glove 2200 of FIG. 21 can exclude one or more of the electrical features 1906, 1908, 1910 mounted to the primary material 2018. Although the non-limiting aspect of FIG. 21 includes a coupling circuit 2210, which can be similarly configured relative to the coupling circuit 1910 of FIG. 19, the glove 2200 can exclude the particular trace configuration 1906 and/or the IMU 1908 of FIG. 19. Rather, the glove 2200 of FIG. 21 can include ten circuits 2204ₐ₋ⱼ, each with a network of elongated, looping traces 2202 mounted to the substrate 2018. According to some non-limiting aspects, the circuits 2204ₐ₋ₑ, including the traces 2202 and substrates 2212, 2218, can be constructed as described in International Patent Application No. PCT/US2022/070853, titled DEVICES, SYSTEMS, AND METHODS FOR MAKING AND USING CIRCUIT ASSEMBLIES HAVING PATTERNS OF DEFORMABLE CONDUCTIVE MATERIAL FORMED THEREIN, and filed Feb. 25, 2022, the disclosure of which is herein incorporated by reference in its entirety. Similar to the traces 1902 of FIG. 19, the traces 2202 of FIG. 21 can include any deformable conductor, such as those disclosed in International Patent Application No. PCT/US2017/019762 titled LIQUID WIRE, which was filed on Feb. 27, 2017 and published on Sep. 8, 2017 as International Patent Publication No. WO2017/151523A1, the disclosure of which is hereby incorporated by reference in its entirety.

Notably, the traces 2202 of FIG. 21 can be particularly configured such that, while wearing the glove 2200, a user's motions can result in deformation of the elongated traces 2202 and/or the coupling circuit 2210 which can alter electrical parameters that can be correlated to baseline data. In the absence of particular trace configurations 1906 of FIG. 19, each circuit 2204ₐ₋ⱼ has a trace 2202 with a desired length. For example, the trace 2202 of the first circuit 2204ₐ, fourth circuit 2204_{d}, sixth circuit 2204_{f}, eighth circuit 2204ₕ, and tenth circuit 2204ⱼ are comparatively shorter than the second circuit 2204_{b}, third circuit 2204_{c}, fifth circuit 2204ₑ, seventh circuit 2204_{g}, and ninth circuit 2204ᵢ. The trace 2202 of the first circuit 2204ₐ, fourth circuit 2204_{d}, sixth circuit 2204_{f}, eighth circuit 2204ₕ, and tenth circuit 2204ⱼ extend to a first location of interest, approximately, where a user's most proximal knuckle of each finger would be positioned. Likewise, the second circuit 2204_{b}, third circuit 2204_{c}, fifth circuit 2204ₑ, seventh circuit 2204_{g}, and ninth circuit 2204ᵢ extend to a second location of interest, approximately, where a user's intermediate knuckle of each finger would be positioned. Accordingly, electrical parameters (e.g., an inductance, a resistance, a voltage drop, a capacitance, and an electromagnetic field, etc.) generated by the traces 2202 of each circuit 2204ₐ, can be compared and correlated to physical parameters (e.g., a strain, a stress, a pressure, a dimension, etc.) associated with one or more portions of the glove 2200 and, thus, can characterize the motion of the user's hand. The differences in correlated physical parameters of each circuit 2204ₐ₋ⱼ can be used to model the user's hand in a virtual environment.

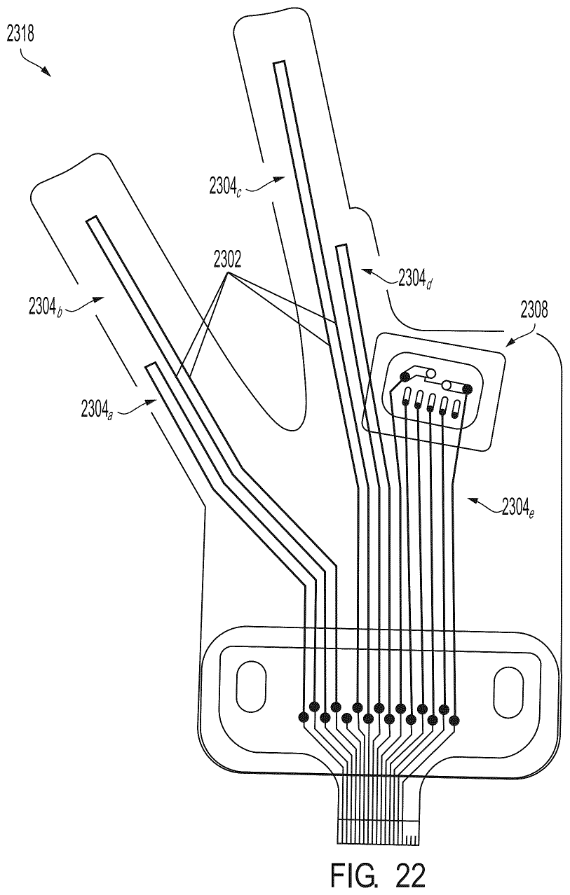

Although the non-limiting aspects of FIGS. 19 and 21 depict gloves 1900, 2200 that include circuits 1904, 2204 with varying trace 1902, 2202 configurations and electrical features 1906, 1908, 1910, 2210, it shall be appreciated that the present disclosure contemplates other non-limiting aspects, featuring a variety of combinations of the previously disclosed trace 1902, 2202 configurations and electrical features 1906, 1908, 1910, 2210. For example, referring now to FIG. 22, another substrate 2318 configured for use with another glove is depicted in accordance with at least one non-limiting aspect of the present disclosure. Once again, FIG. 22 illustrates the modular construction of another glove contemplated by the present disclosure. However, according to the non-limiting aspect of FIG. 22, the substrate 2318 can be constructed with a different circuit 2304_{a-c} configuration than previously discussed.

In further reference to FIG. 22, the substrate 2318 can be similar in construction to the substrate 1918 of FIGS. 19 and 20, featuring a first circuit 2304a a second circuit 2304_{b}, a third circuit 2304_{c}, and a fourth circuit 2304_{d}, each configured to traverse one of the user's thumb or pointer finger, when wearing a glove with the substrate 2318 mounted to it. The substrate further includes a fifth circuit 2304ₑ that includes an IMU 2308, which can be positioned in the palm of the glove and configured similar to the IMU 1908 of FIG. 19. However, unlike the substrate 1918 of FIGS. 19 and 20, the substrate 2318 of FIG. 22 excludes the particular trace configurations 1906 (FIGS. 19 and 20) and, rather, can include elongated, looping traces 2302.

It shall be noted that the circuits 2304_{a-d} of the substrate 2318 of FIG. 22 can extend to locations of interest, similarly to the traces 2204 of FIG. 21. Specifically, the traces 2304 of the first circuit 2304a and fourth circuit 2304_{d} extend to a first place of interest, approximately, where a user's proximal knuckle of the thumb and pointer finger would be positioned. The traces 2304 of the second circuit 2304_{b} and third circuit 2304_{c} extend to a second place of interest, approximately, where a user's intermediate knuckle of the thumb and pointer finger would be positioned. Accordingly, electrical parameters (e.g., an inductance, a resistance, a voltage drop, a capacitance, and an electromagnetic field, etc.) generated by the traces 2304 of each circuit 2304ₐ₋ₑ can be compared and correlated to physical parameters (e.g., a strain, a stress, a pressure, a dimension, etc.) associated with one or more portions of the glove 2300 and, thus, can characterize the motion of the user's hand. In conjunction with signals generated by the IMU 2308, which can be configured similarly to the IMU 1908 of FIG. 19, the traces can generate signals which correlate to physical parameters of each circuit 2304ₐ₋ₑ to model the user's hand in a virtual environment.

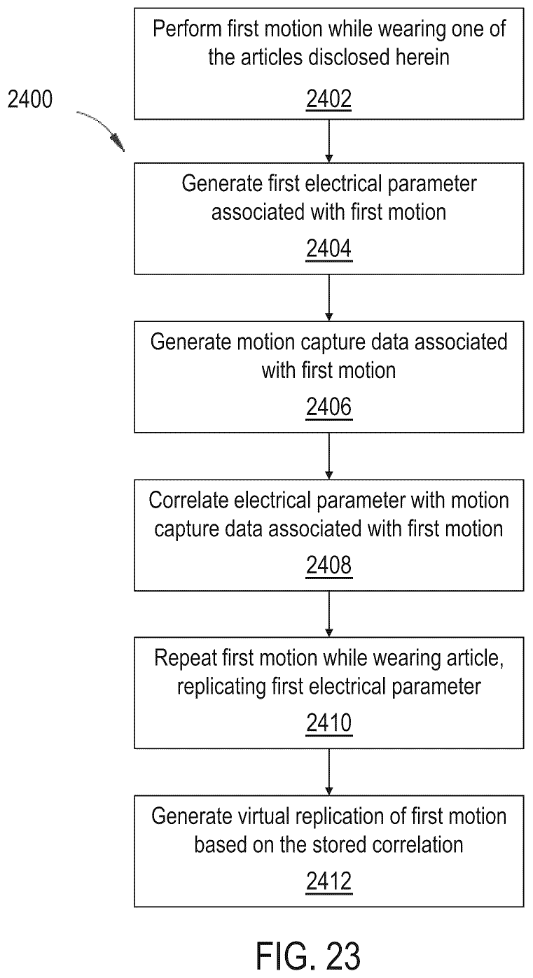

Referring now to FIG. 23, a method 2400 of generating signals associated with electrical parameters and correlating those electrical parameters to the physical motions of a user of the gloves disclosed herein is depicted in accordance with at least one non-limiting aspect of the present disclosure. According to the non-limiting aspect of FIG. 23, the method 2400 can include performing 2402 a first motion while wearing one of the articles disclosed herein. Upon performing 2402 the first motion, one of the flexible circuits can generate a first electrical parameter (e.g., an inductance, a resistance, a voltage drop, a capacitance, and an electromagnetic field, etc.) associated with the first motion via any of the trace configurations and/or electrical features disclosed herein. The first motion can be monitored via a camera, or any other device capable of generating 2406 motion capture data associated with the first motion. Once the electrical parameter and motion capture data associated with the first motion are generated, the electrical parameter associated with the first motion can be correlated 2408 to the motion capture data associated with the first motion. The correlation can be stored such that, when the first motion is repeated 2410, a processor communicably coupled to the articles disclosed will receive one or more signals that it can determine are associated with the first electrical parameter. Accordingly, the processor can generate 2412 a virtual replication of the first motion based on the stored correlation.

However, the steps illustrated in FIG. 23 are not the exclusive steps of the method 2400 contemplated by the present disclosure. For example, according to some non-limiting aspects, the method 2400 can further include generating baseline electrical parameters and replicating the steps for a plurality of motions, such that an entire range of motions can be virtually replicated using the articles disclosed herein. According to some non-limiting aspects, the method can include the interim step of correlating the electrical parameter to a physical parameter (e.g., a strain, a stress, a pressure, a dimension, etc.) of the article and its circuits. In some non-limiting aspects, correlating the electrical parameter to the physical parameter can occur in lieu of correlating the electrical parameter to the motion capture data. Moreover, the method can include receiving and processing input from one or more pressure sensors coupled to the article and virtually recreating an interaction between a user of the article and an object in the real environment, based on signals received from the one or more pressure sensors.

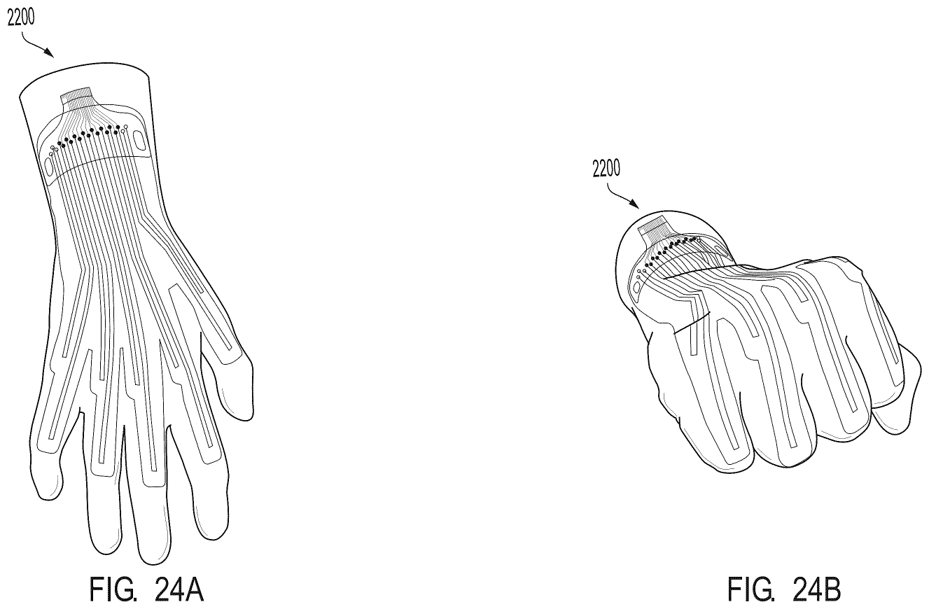

Referring now to FIGS. 24A and 24B, the glove 2200 of FIG. 21 is depicted in use via the method 2400 of FIG. 23, according to at least one non-limiting aspect of the present disclosure. For example, according to FIG. 24A, the user's hand is relaxed while using the glove 2200. Accordingly, a processor can generate and record signals received from the circuits 2204ₐ, (FIG. 21) when the glove 2200 is in the first, relaxed position of FIG. 24A. A device capable of generating motion capture data, such as a camera, can be used to record the glove 2200 as the user flexes their hand into a fist, as depicted in FIG. 24B. The processor can subsequently generate and record signals received from the circuits 2204ₐ, (FIG. 21) when the glove 2200 is in the second, flexed position of FIG. 24B. The processor can correlate the electrical parameter associated with the first, relaxed position of FIG. 24A with the motion capture data associated with the first, relaxed position of FIG. 24A, and the electrical parameter associated with the second, flexed position of FIG. 24B with the motion capture data associated with the second, flexed position of FIG. 24B. Accordingly, the processor can generate a virtual simulation of the user's hand as it transitions from the first, relaxed position of FIG. 24A to the second, flexed position of FIG. 24B, every time the user performs the motion, based on electrical parameters received from the glove 2200 alone, without the assistance of real-time motion capture data generated by a camera.

It shall be appreciated that wearable articles, such as the glove 2200 of FIG. 21, can be used to simulate the motions of user in a virtual environment. This can provide numerous benefits due to a reduction of ancillary components required to simulate the user's motions while in use. For example, conventional articles may rely on a plurality of IMUs, gyroscopes, and/or accelerometers to estimate the articles position and/or orientation in space. However, such components can be bulky and/or uncomfortable for the user and may have increased requirements causing the article to be impractical and inefficient for everyday use. As such, there is a need for devices, systems, and methods for simulating motions in a virtual environment using a wearable article with flexible circuits. The flexible circuits can reduce the number of ancillary components needed to simulate the user's motions in a virtual environment and thus, can result in a more streamlined fit that requires less power to achieve the same, or enhanced results.

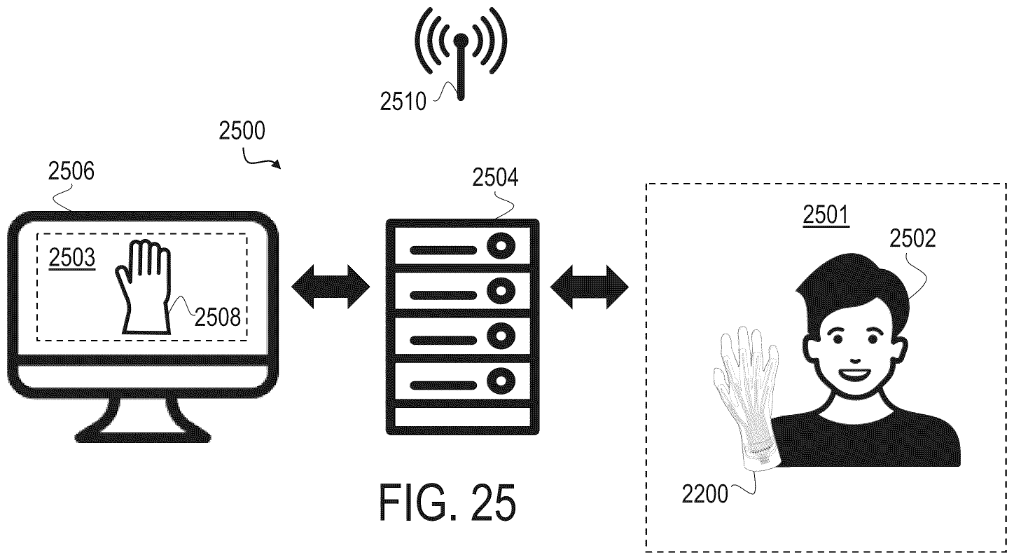

Referring now to FIG. 25, a system 2500 configured to simulate motions in a virtual environment using a wearable article with flexible circuits, according to at least one non-limiting aspect of the present disclosure. According to the non-limiting aspect of FIG. 25, the system 2500 can include a wearable article, such as the glove 2200 of FIG. 21, a computing device, such as a server 2504, and a display 2506. Of course, according to other non-limiting aspects, the computing device can include any other device capable of receiving, processing, and outputting signals, such as a personal computer, a laptop computer, a tablet, mobile computing device (e.g., a smart phone, smart glasses, a virtual reality or augmented reality headset, etc.), and/or a hobbyist computing device (e.g., Arduino®, Raspberry Pi®, etc.), amongst others. According to still other non-limiting aspects, the display 2506 can be integral to the computing device (e.g., a laptop, a smart phone, etc.), or integral to an ancillary computing device communicably coupled to the computing device depicted in FIG. 25. For example, according to some non-limiting aspects, the display 2506 can be a smart phone or a virtual reality or augmented reality headset communicably coupled to the server 2504 and, thus, a user can remotely view and/or interact with the generated simulation 2508 more conveniently while the server 2504 performs the requisite processing functions.

The glove 2200, server 2504, and display 2506 can be communicably coupled via any wired and/or wireless connection. For example, according to the non-limiting aspect of FIG. 25, the system 2500 can further include a wireless access point 2510 configured to communicably couple at least two of the glove 2200, the server 2504, and/or the display 2506. However, according to other non-limiting aspects, at least the glove 2200 is communicably coupled to the server 2504 via a serial communications connection (e.g., universal serial bus, serial peripheral interface, RS-type connectors, etc.) and/or protocol (e.g., Modbus®, open platform, etc.), or some other means of transmitting signals-such as those associated with electrical parameters generated by the flexible circuits—to and/or from the glove 2200.

As previously discussed, the computing device can include a server 2504 or any other device capable of receiving, processing, and outputting signals generated by the glove 2200 or any other wearable article that utilizes flexible circuits similar to those described herein. According to the non-limiting aspect of FIG. 25, the server 2504 can include a memory and a control circuit, such as a processor or microprocessor configured to execute instructions stored in the memory. The server 2504 can be configured to store software or firmware configured to enable the server 2504 to communicate data to and/or from the glove 2200 of the system 2500. For example, according to some non-limiting aspects, the server 2504 can be configured to store a SerialCom® plugin that enables the transmission of custom data packages to and/or from the glove 2200 or any other wearable article. In other words, signals transmitted by the glove 2200 can include custom data packages that, for example, can include ten data points, each corresponding to an electrical parameter generated by each circuit 2204ₐ, (FIG. 21) mounted to a substrate 2018 (FIG. 21) of the glove 2200.

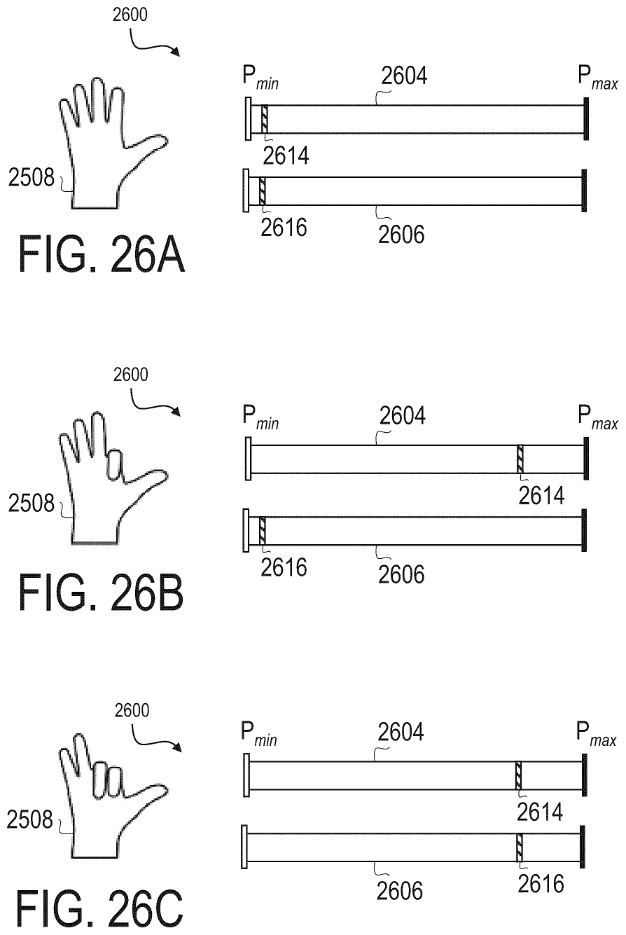

In further reference to FIG. 25, the server 2504 can be further configured to store a visualization engine. According to some non-limiting aspects, the visualization engine can include a commercially available platform, such as the Unreal Engine®, the GoDot® engine, the Unity® engine, the GDevelop® engine, the CRYENGINE®, and/or the Verge3D® engine, amongst others. According to other non-limiting aspects, the visualization engine can include a custom build. Regardless, the visualization engine can include an input system configured to convert user inputs-such as signals associated with electrical parameters generated by flexible circuits of the glove 2200—into simulated actions performed by an avatar 2508 in the virtual environment 2503. This input system can be configured through a simulation framework, which will be described in further detail in reference to FIGS. 26A-C. In summary, the simulation framework can include rules by which the simulation is generated and updated by the visualization engine executed by the server 2504 or any other processor communicably coupled to the glove 2200. In other words, by employing a simulation framework, the visualization engine can track the motions of a user wearing the glove 2200 in a physical environment 2501 and simulate those motions via an avatar 2508 in a virtual environment 2503. The avatar 2508 can include a custom build or can include an imported model from a third party (e.g., MakeHuman®, Maximo®, etc.).

Referring now to FIGS. 26A-C, a simulation framework 2600 configured to be run via the system 2500 of FIG. 25 is depicted in accordance with at least one non-limiting aspect of the present disclosure. According to the non-limiting aspect of FIGS. 26A-C, the simulation framework 2600 can include a plurality of scales 2604, 2606, wherein each scale 2604, 2606 corresponds to each sensor 2204ₐ, (FIG. 21) of the glove 2200 (FIGS. 21 and 25). Each scale 2604, 2606 can be defined by a minimum electrical parameter Pₘᵢₙ and a maximum electrical parameter Pₘₐₓ associated with each sensor 2204ₐ₋ⱼ, (FIG. 21) of the glove 2200 (FIGS. 21 and 25). For example, the simulation framework employed by the visualization engine executed by the system 2500 of FIG. 25 can assess electrical parameters (e.g., an inductance, a resistance, a voltage drop, a capacitance, and an electromagnetic field, etc.) generated by the sensing circuits 2204ₐ, (FIG. 21) of the glove 2200 (FIGS. 21 and 25) relative to the scale 2604, 2606 corresponding to each particular sensing circuit 2204ₐ, (FIG. 21). For purposes of illustration, the scales 2604, 2606 of FIGS. 26A-C correspond to one or more sensing circuits 2204_{c-f}(FIG. 21) positioned on a pointer finger and middle finger of the glove 2200 (FIGS. 21 and 25).

However, according to some non-limiting aspects, the minimum electrical parameter Pₘᵢₙ and a maximum electrical parameter Pₘₐₓ for each scale 2604, 2606 can differ for each sensor 2204ₐ, (FIG. 21) of the glove 2200 (FIGS. 21 and 25), as the minimum electrical parameter Pₘᵢₙ and a maximum electrical parameter Pₘₐₓ may differ according to certain variables associated with each sensor 2204ₐ, (FIG. 21). For example, the minimum electrical parameter Pₘᵢₙ and a maximum electrical parameter Pₘₐₓ may vary depending on what finger 2204ₐ₋ⱼ (FIG. 21) the sensor is positioned on and/or whether or not the sensor 2204ₐ, (FIG. 21) runs to the knuckle or the fingertip. Although FIGS. 26A-C show two scales 2604, 2606 for illustrative purposes, it shall be appreciated that the framework can include similar scales for each sensor 2204ₐ, (FIG. 21) of the glove 2200 (FIGS. 21 and 25).

According to the non-limiting aspect of FIGS. 26A-C, the visualization engine can receive signals associated with electrical parameters 2614, 2616 generated by one or more sensing circuits 2204_{c-f}(FIG. 21) positioned on a pointer finger and a middle finger of the glove 2200 (FIGS. 21 and 25) in real time. For example, according to FIG. 26A, the visualization engine can assess, via the simulation framework 2600, that the received electrical parameters 2614, 2616 are relatively close to the minimum electrical parameter Pₘᵢₙ of each scale 2604, 2606. Accordingly, the visualization engine can correlate the received electrical parameters 2614, 2616 with a physical condition of the sensing circuits 2204_{c-f}(FIG. 21) positioned on a pointer finger and a middle finger of the glove 2200 (FIGS. 21 and 25). For example, based on the correlation, the visualization engine may determine that the minimum electrical parameter Pₘᵢₙ occurs when the sensing circuits 2204_{c-f}(FIG. 21) positioned on a pointer finger and a middle finger of the glove 2200 (FIGS. 21 and 25) are experiencing little or no strain. As such, the visualization engine may determine that because the sensing circuits 2204_{c-f}(FIG. 21) are not strained, the user is not flexing their pointer finger or middle finger. Thus, the visualization engine will generate a simulation wherein the avatar 2508 has an open palm, as depicted in FIG. 26A.

According to the non-limiting aspect of FIG. 26B, the visualization engine can further assess, via the simulation framework 2600, that an electrical parameter 2614 associated with a signal generated by one or more sensing circuits 2204_{c}, 2204_{d} (FIG. 21) positioned on a pointer finger of the glove 2200 (FIGS. 21 and 25) is relatively close to the maximum electrical parameter Pₘₐₓ of the scale 2604. The visualization engine can correlate the received electrical parameters 2614, 2616 with a physical condition of the sensing circuits 2204_{c-f} (FIG. 21). However, according to the non-limiting aspect of FIG. 26B and based on the correlation, the visualization engine may determine that the maximum electrical parameter Pₘₐₓ occurs when the sensing circuits 2204_{c-f}(FIG. 21) are experiencing a maximum amount of strain. As such, the visualization engine may determine that because one or more sensing circuits 2204_{c}, 2204_{d} (FIG. 21) on the pointer finger of the glove 2200 (FIGS. 21 and 25) are strained, the user is thus flexing the pointer finger. Once again, the visualization engine may determine that, because one or more sensing circuits 2204ₑ, 2204_{f} (FIG. 21) on the middle finger are experiencing little or no strain, the user is not flexing their middle finger. Thus, the visualization engine will generate a simulation wherein the avatar 2508 has curled their pointer finger, but otherwise maintains an open palm, as depicted in FIG. 26B.

According to the non-limiting aspect of FIG. 26C, the visualization engine can further assess, via the simulation framework 2600, that electrical parameters 2614, 2616 associated with signals generated by the one or more sensing circuits 2204_{c-f} (FIG. 21) positioned on a pointer finger and middle finger of the glove 2200 (FIGS. 21 and 25) are relatively close to the maximum electrical parameter Pₘₐₓ of the scales 2604, 2606. The visualization engine can correlate the received electrical parameters 2614, 2616 with a physical condition of the sensing circuits 2204_{c-f} (FIG. 21). However, according to the non-limiting aspect of FIG. 26C and based on the correlation, the visualization engine may determine that, because one or more sensing circuits 2204_{c-f} (FIG. 21) on the pointer and middle fingers are strained, the user is flexing both the pointer and middle finger. This is because the maximum electrical parameter Pₘₐₓ occurs when the sensing circuits 2204_{c-f}(FIG. 21) are experiencing a maximum amount of strain. Thus, the visualization engine will generate a simulation wherein the avatar 2508 has curled the pointer and middle finger, but otherwise maintains an open palm, as depicted in FIG. 26C.

It shall be appreciated that there are intermediate positions that are not depicted in FIGS. 26A-C that are important to the overall simulation of the motions of the user when wearing the glove 2200 (FIGS. 21 and 25). In other words, the rate at which the avatar 2508 can be updated to reflect the user's motions can be as frequent as signal generation by the glove 2200 (FIGS. 21 and 25) and refresh rate of the display 2506 will allow. Signals can be generated by the glove 2200 (FIGS. 21 and 25) in real-time, and the refresh rate of the display can range anywhere from 60 Hz to 240 Hz. Although in many applications, it may be beneficial to have virtual real-time simulation of the motions of the user when wearing the glove 2200, (FIGS. 21 and 25) any rate of update can be implemented by the system 2500 of FIG. 25. For example, according to some non-limiting aspects, the avatar 2508 can be updated only when certain positions or milestones are achieved.

Although not depicted in FIGS. 26A-C, it shall be appreciated that the use of two or more sensors on each finger can further enhance the simulation. For example, by receiving and comparing signals associated with electrical parameters generated by sensing circuits that extend to a user's knuckle (e.g., sensing circuits 2204_{d}, 2204_{f} of FIG. 21, etc.) relative to signals associated with electrical parameters generated by sensing circuits that extend to a user's fingertip (e.g., sensing circuits 2204_{c}, 2204ₑ of FIG. 21, etc.), the framework 2600 of FIGS. 26A-C can determine the amount of flexion a finger has, with precision. For example, if the visualization determines that signals associated with electrical parameters generated by sensing circuits that extend to a user's fingertip (e.g., sensing circuits 2204_{c}, 2204ₑ of FIG. 21, etc.) are closer to a maximum electrical parameter Pₘₐₓ than a minimum electrical parameter Pₘᵢₙ and that signals associated with electrical parameters generated by sensing circuits that extend to a user's knuckle (e.g., sensing circuits 2204_{d}, 2204_{f} of FIG. 21, etc.) are closer to a minimum electrical parameter Pₘᵢₙ than a maximum electrical parameter Pₘₐₓ, then the visualization engine may determine that most of the user's finger is straight, but slightly bent towards the fingertip. Obviously, the degree of precision associated with the generated simulation will scale proportionally with the relative magnitude of received electrical parameters 2614, 2616. In other words, the relative rotation about various knuckles in the user's hand can be accurately simulated in a way conventional devices are incapable of efficiently simulating.

Furthermore, it shall be appreciated that degree by which a finger has flexed or curled is only one degree of motion that can be simulated via the framework 2600 of FIGS. 26A-C and system 2500 of FIG. 25. For example, data generated by all of the sensing circuits 2204ₐ₋ⱼ (FIG. 21) can be correlated to data associated with physical conditions of each of the sensing circuits 2204ₐ₋ⱼ (FIG. 21) and compared relative to one another, such that a distance between fingers, or “splay,” can be accurately simulated by the visualization engine. Likewise, a rotation and/or transverse motion of each finger, including the thumb, can be determined by the visualization engine via the framework and simulated via the avatar 2508.

According to non-limiting aspects wherein the glove 2200 (FIGS. 21 and 25) further includes one or more IMUs, data generated by the sensing circuits 2204ₐ₋ⱼ (FIG. 21) can be further used in conjunction with data generated by the IMUs to enhance the generated simulation via the avatar 2508. In still other non-limiting aspects, the data from the sensing circuits 2204ₐ₋ⱼ (FIG. 21) can be used to calibrate on-board IMUs and, thus, eliminate IMU drift. For example, one such aspect contemplates two IMUs positioned on the glove 2200 (FIGS. 21 and 25) with a sensing circuit positioned between the IMUs. Here, data from the intermediate sensing circuit can be utilized to correct data generated by either IMU and, thus, enhance the simulated position and orientation of the avatar 2508 in the virtual environment 2503 (FIG. 25).

It shall be appreciated that, although the present disclosure discusses correlating data generated by the sensing circuits 2204ₐ₋ⱼ (FIG. 21) to motion capture data, data generated by the sensing circuits 2204ₐ₋ⱼ (FIG. 21) can be correlated to any other data that assists in characterizing the physical condition of each of the sensing circuits 2204ₐ₋ⱼ (FIG. 21). For example, measurements can be taken manually, distances can be estimated based on still photos, or video can be utilized by the user to enhance the matrices of data used by the visualization engine to correlate data generated by the sensing circuits 2204ₐ₋ⱼ (FIG. 21) and enhance the accuracy of the simulation via the avatar 2508.

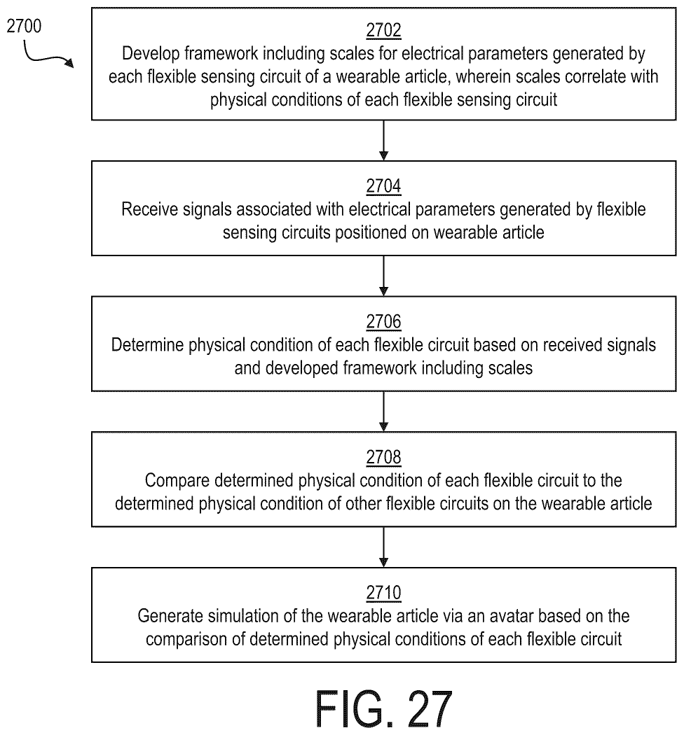

Referring now to FIG. 27, a method 2700 of simulating motions in a virtual environment 2503 (FIG. 25) using a wearable article with flexible circuits is depicted in accordance with at least one non-limiting aspect of the present disclosure. The method can be performed, for example, via the system 2500 of FIG. 25. According to the non-limiting aspect of FIG. 27, the method 2700 can include developing 2702 a framework that includes scales for electrical parameters generated by each flexible sensing circuit of a wearable article, wherein scales correlate with physical conditions of each flexible sensing circuit. For example, the framework can be similar to the framework 2600 of FIGS. 26A-C, and data associated with the physical conditions of the flexible circuits can be generated via the method 2400 of FIG. 23. However, according to other non-limiting aspects, data associated with the physical conditions of the flexible circuits can be taken manually, distances can be estimated based on still photos, and/or video can be utilized by the user to generate data that can be correlated with electrical parameters generated by the flexible circuits.

Still referring to FIG. 27, the method 2700 can further include receiving 2704 signals associated with electrical parameters generated by flexible sensing circuits positioned on a wearable article. The signals can be generated as the user wears the wearable article and moves around a physical environment 2501 (FIG. 25). Once received, the method 2700 can include determining 2706 a physical condition of each flexible circuit based on the received signals. As depicted in FIGS. 26A-C, the determination 2706 can be further based on a developed framework 2600 that includes scales 2604, 2606 that correspond to each of the flexible circuits on the wearable article. After determining 2706 the physical condition of the flexible circuits, the method 2700 can include comparing 2708 each determined physical condition of each flexible circuit to the determined physical condition of other flexible circuits on the wearable article. This comparison 2708 can provide the visualization engine with the relative information it needs to generate the simulation. Accordingly, the method 2700 can further include generating 2710 the simulation of the wearable article via an avatar based on the comparison of determined physical conditions of each flexible circuit.

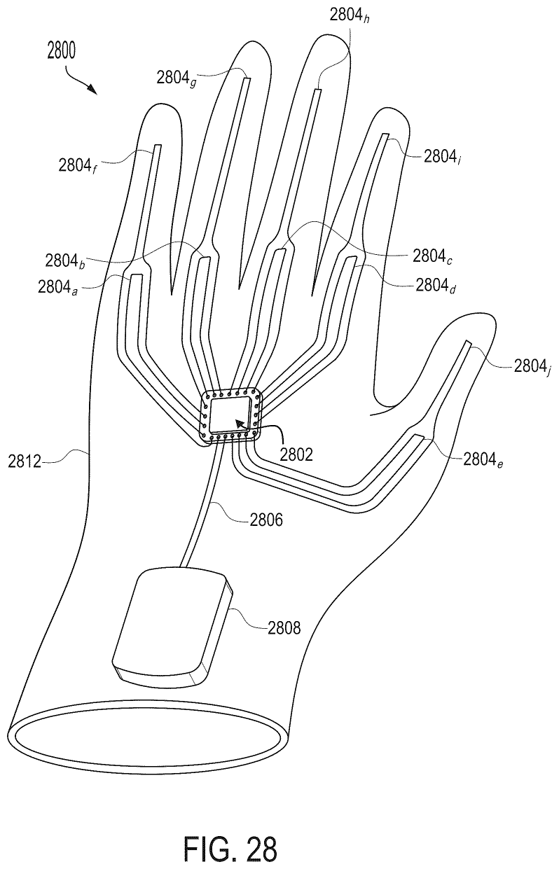

Referring now to FIG. 28, another article 2800 configured for the simulation of physical motions in a virtual environment is depicted in accordance with at least one non-limiting aspect of the present disclosure. Similar to the articles 1900, 1920 of FIGS. 19 and 22, the article 2800 can be configured as a glove to be worn on a user's hand. Once again, the glove 2800 can include certain elements that apply the aforementioned principles and techniques to generate electrical parameters, which can be correlated to physical parameters associated with a user's physical movements, when wearing the glove. Of course, according to other non-limiting aspects, the article can take the form of any other article of clothing, including a knee glove, a shirt, pants, a sock, and/or a hat, amongst others.

In further reference to FIG. 28, the glove 2800 can include a first plurality of flexible circuits 2804ₐ₋ₑ and a second plurality of flexible circuits 2804_{f-j} positioned on the back-hand side of the glove 2800 when worn by the user, each of which can be composed of a deformable conductor, as described herein, and each of which can be arranged and positioned to monitor and characterize a different part of the user's hand when the glove 2800 is worn. Unlike the flexible circuits 1904ₐ₋ₑ of FIG. 19, the flexible circuits 2804ₐ₋ⱼ of FIG. 28 do not have electrical features but instead are arranged in a simple “U” shaped configuration. According to some non-limiting aspects, the first and second plurality of circuits 2804ₐ₋ⱼ can include traces and substrates, such as those described in International Patent Application No. PCT/US2022/070853, titled DEVICES, SYSTEMS, AND METHODS FOR MAKING AND USING CIRCUIT ASSEMBLIES HAVING PATTERNS OF DEFORMABLE CONDUCTIVE MATERIAL FORMED THEREIN, and filed Feb. 25, 2022, the disclosure of which is herein incorporated by reference in its entirety.

For example, according to the non-limiting aspect of FIG. 28, the first plurality of flexible circuits 2804ₐ₋ₑ can be shorter in length than the second plurality of flexible circuits 2804_{f-j} and can be arranged such that each of the first plurality of flexible circuits 2804ₐ₋ₑ extends over—and terminates just beyond—the metacarpophalangeal, or most proximal, knuckles of each finger of the user when the glove 2800 is worn. The second plurality of circuits 2804_{f-j}, for example, can be longer in length than the first plurality of flexible circuits 2804ₐ₋ₑ and can be arranged such that each of the second plurality of flexible circuits 2804_{f-j} extends over the proximal and distal interphalangeal joints of each finger when the glove 2800 is worn. In other words, unlike previously described aspects of the present disclosure, the second plurality of flexible circuits 2804_{f-j} can be configured to extend beyond the intermediate knuckles and over both of the phalangeal knuckles for a more precise monitoring of motions. According to the non-limiting aspect of FIG. 28, the longer, second plurality of circuits 2804_{f-j} can surround the shorter, first plurality of flexible circuits 2804ₐ₋ₑ.

According to the non-limiting aspect of FIG. 28, the first plurality of flexible circuits 2804ₐ₋ₑ and the second plurality of flexible circuits 2804_{f-j} can be terminated at an island 2802 positioned approximately halfway down the user's hand on the back-hand side of the glove 2800 when worn by the user. Thus, motions of the user's fingers, exclusively, can be targeted for monitoring without interference from motions of the wrist. According to some non-limiting aspects, the island 2802 can include an ADC configured to convert the analog signals generated by each flexible circuit 2804ₐ₋ⱼ into a digital signal and transmit the resulting digital signals via a bus circuit 2806 configured for power and/or data transmission. According to other non-limiting aspects, the island 2802 can include a processor configured to process the analog signals and package them into a consolidated digital signal that includes digital signals representing electrical parameters generated by each of the flexible circuits 2804ₐ₋ⱼ.

Similarly to the electrical features 1908, 1910, 1920 described in reference to FIG. 19, it shall be appreciated that island 2802 of FIG. 28 can include a PCB construction including a polyimide flexible board construction that can be bonded to the laminate structure. Electrical contacts (not shown) and/or traces (not shown) of the island 2802 can be reflow soldered to the flexible PCB. For example, according to some non-limiting aspects, the PCBs can be constructed as described in U.S. patent application Ser. No. 16/885,854, titled CONTINUOUS INTERCONNECTS BETWEEN HETEROGENEOUS MATERIALS, and filed May 28, 2020, the disclosure of which is herein incorporated by reference in its entirety. Alternatively and/or additionally, various chips and/or sensors can be directly hosted on the laminate circuit structure itself.

According to some non-limiting aspects, the island 2802 of the glove of FIG. 28 can include an IMU that, in conjunction with signals generated by one or more other circuits 2804ₐ₋ⱼ, can be correlated to physical parameters of the glove 2800 and used to characterize a user's motions when wearing the glove 2800. For example, it shall be appreciated that an IMU can include a number of accelerometers, which can output linear acceleration signals on three axes in space, and/or gyroscopes, which can output angular velocity signals on three axes in space, to measure triaxial acceleration and/or angular velocity of the user's hand while wearing the glove 2800. It shall be further appreciated how, in conjunction with the other circuits 2804ₐ₋ⱼ, the IMU can be used to determine other aspects of a POSE of the glove 2800 in three-dimensional space. For example, the various circuits 2804ₐ₋ⱼ can generate electrical parameters (e.g., an inductance, a resistance, a voltage drop, a capacitance, and an electromagnetic field, etc.), which can be used to contextualize and/or calibrate signals generated by the IMU. Accordingly, if the IMU begins to drift due to extended use, a processor communicably coupled to the circuits 2804ₐ₋ⱼ can utilize signals associated with electrical parameters from the other circuits 2804ₐ₋ⱼ to correct signals received from the IMU.

Still referring to FIG. 28, according to some non-limiting aspects, the bus circuit 2806 can also include traces composed of the deformable conductors described herein and can be used to monitor other motions of the user's hand while wearing the glove 2800. For example, according to such aspects, the bus circuit 2806 can be used to specifically monitor the motions of a user's wrist. Nonetheless, the bus circuit 2806 can transmit digital signals to and/or from an electronic component 2808 positioned on the glove 2800. According to some non-limiting aspects, the electronic component 2808 can be configured similarly to the power components described in U.S. Provisional Patent Application No. 63/412,867, titled DEVICES, SYSTEMS, AND METHODS TO MONITOR AND CHARACTERIZE THE MOTIONS OF AUSER VIA FLEXIBLE CIRCUITS, and filed on Oct. 3, 2022, the disclosure of which is hereby incorporated by reference in its entirety.

According to some non-limiting aspects, the electronic component 2808 of the glove 2800 of FIG. 28 can be configured for onboard signal processing and/or transmission. For example, according to some non-limiting aspects, the electronic component 2808 can include a microprocessor (e.g., a Nordic-brand nRF MDK-based processor or equivalent, etc.), a memory, a wireless communication circuit, and/or a bus port (configured to receive power and/or data from a power component of the electronic component 2808), an additional IMU, and/or additional sensors, amongst other electronic components. According to some non-limiting aspects, the aforementioned ADC, for example, can be positioned on the electronic component 2808. According to still other non-limiting aspects, the electronic component 2808 can include an electrode, such as any of the electrodes described herein.

According to other non-limiting aspects, the electronic component 2808 can include a power source, such as a battery and/or a charger. The charger, for example, can include a USB port configured to convey electrical power and/or data to the electronic component 2808 from an external source. For example, the electronic component 2808 can be configured for such conveyance via a USB-A. USB-B, or USB-C protocol, although other means for power and/or data conveyance are contemplated by the present disclosure. According to other non-limiting aspects, the electronic component 2808 can include a wireless charging circuit and/or a wireless transmitter and/or receiver configured to wirelessly obtain power and data from external sources. Regardless, it shall be appreciated that the electronic component 2808, when mechanically and electrically coupled to the glove 2800, can provide electrical power to the island 2802 and/or flexible circuits 2804ₐ₋ⱼ. Additionally, via the electronic component 2808, it shall be appreciated that data can be transmitted to and from the island 2802 and/or flexible circuits 2804ₐ₋ⱼ. For example, according to some non-limiting aspects, the electronic component 2808 can be used to transmit a firmware update to a memory of the island 2802 for execution by a microprocessor. Alternately, the electronic component 2808 can include a memory configured to store data generated by the flexible circuits 2804ₐ₋ⱼ for subsequent use and processing.

According to still other non-limiting aspects, the glove 2800 can include a mechanical component, such as a cradle, configured to removably secure the electronic component 2808 to the glove 2800. Accordingly, the cradle can establish electrical communication between the electronic component 2808 and the bus circuit 2806, thereby enabling the electronic component 2808 to power the circuits 2804ₐ₋ⱼ, 2806 and island 2802 of the glove 2800 of FIG. 28. According to still other non-limiting aspects, the electronic component 2808 can include a memory and/or transceiver. Thus, when the electronic component 2808 is mechanically secured to the wearable article 2800 via the cradle, the electronic component 2808 can provide power and/or data to the other electronics of the glove 2800.

It shall be appreciated that one or more of the components (e.g., microprocessor, memory, wireless circuit, ADC, IMU, other sensors, etc.) of the island 2802 of FIG. 28 can be alternately positioned within the electronic component 2808. Accordingly, via the electronic component 2808, some or all of the functionality provided by the island 2802 can be modular and interchangeable amongst several flexible circuits and/or wearable articles. This can promote efficiency and reduce the expense associated with manufacturing the wearable article 2800 itself, According to some non-limiting aspects, the electronic component 2808 can include an RFID chip or another means of identifying its identity. Accordingly, the electronic component 2808 can be associated with and/or linked to a particular wearable article, such as the glove 2800 of FIG. 28. This can ensure accurate tagging of data, including the association of data with a specific user.

Accordingly, the glove 2800 of FIG. 28 can include a circuit 2804ₐ₋ⱼ, 2806 configuration that is more efficient, allows for better packaging, and more exclusively monitors the finger movement with minimum flexions caused by motions of the wrist.

Since the inventive principles of this patent disclosure can be modified in arrangement and detail without departing from the inventive concepts, such changes and modifications are considered to fall within the scope of the following claims. The use of terms such as “first” and “second” are for purposes of differentiating different components and do not necessarily imply the presence of more than one component.

Various aspects of the subject matter described herein are set out in the following numbered clauses:

Clause 1: A system configured to simulate a physical motion performed by a user via an avatar in a virtual environment, the system including a wearable article communicably including a first flexible circuit, wherein the first flexible circuit includes a first trace including a deformable conductor, wherein the first flexible circuit is positioned in a first location of interest on the wearable article, and a computing device including a processor and a memory configured to store a visualization engine that, when executed by the processor, causes the processor to receive a first signal generated by the first flexible circuit, determine a first electrical parameter based on the first signal, scale the first electrical parameter based on a predetermined simulation framework of the visualization engine, wherein scaling the first electrical parameter corresponds to a physical condition of the first flexible circuit, compare the physical condition of the first flexible circuit to previously determined physical conditions associated with the wearable article, and generate a simulation of the physical motion performed by a user via the avatar in the virtual environment based on the comparison.

Clause 2. The system according to clause 1, wherein the wearable article further includes an inertial measurement unit (“IMU”) configured to monitor a position and orientation of the wearable article in three-dimensional space, and wherein, when executed by the processor, the visualization engine further causes the computing device to receive a second signal generated by the IMU, and wherein generation of the simulation is further based on the second signal received from the IMU.

Clause 3. The system according to either clause 1 or clause 2, wherein, when executed by the processor, the visualization engine further causes the computing device to calibrate the second signal generated by the IMU based on the first signal generated by the first flexible circuit.

Clause 4. The system according to any of clauses 1-3, wherein the computing device is positioned remotely relative to the wearable article.

Clause 5. The system according to any of clauses 1-4, wherein the wearable article further includes a transceiver configured to transmit signals to and from the computing device.

Clause 6. The system according to any of clauses 1-5, further including an electronic component including a power source configured to provide electrical power to the first flexible circuit, and wherein the wearable article further includes a mechanical component configured to selectively receive the electronic component.

Clause 7. The system according to any of clauses 1-6, wherein the electronic component further includes a memory configured to store data associated with the first signal generated by the first flexible circuit.

Clause 8. The system according to any of clauses 1-7, wherein the wearable article further includes a second flexible circuit, wherein the second flexible circuit includes a second trace including a deformable conductor, and wherein the second flexible circuit is positioned in a second location of interest on the wearable article.

Clause 9. The system according to any of clauses 1-8, wherein the wearable article is a glove, and wherein the first location of interest includes a most proximal knuckle of a first finger of the glove.

Clause 10. The system according to any of clauses 1-9, wherein the second location of interest includes an intermediate knuckle of the first finger.

Clause 11. The system according to any of clauses 1-10, wherein the second location of interest further includes a most distal knuckle of the first finger.

Clause 12. The system according to any of clauses 1-11, wherein the second flexible circuit traverses around the first flexible circuit.

Clause 13. The system according to any of clauses 1-12, wherein the wearable article further includes a third flexible circuit, wherein the third flexible circuit includes a third trace including a deformable conductor, wherein the third flexible circuit is positioned in a third location of interest on the wearable article, and wherein the third location of interest includes a second finger of the glove.

Clause 14. A wearable article configured to simulate a physical motion performed by a user via an avatar in a virtual environment, the wearable article including a first flexible circuit, wherein the first flexible circuit includes a first trace including a deformable conductor, wherein the first flexible circuit is positioned in a first location of interest on the wearable article, and a circuit configured to communicably couple the first flexible circuit to a computing device including a processor and a memory configured to store a visualization engine that, when executed by the processor, causes the processor to receive a first signal generated by the first flexible circuit, determine a first electrical parameter based on the first signal, scale the first electrical parameter based on a predetermined simulation framework of the visualization engine, wherein scaling the first electrical parameter corresponds to a physical condition of the first flexible circuit, compare the physical condition of the first flexible circuit to previously determined physical conditions associated with the wearable article, and generate a simulation of the physical motion performed by a user via the avatar in the virtual environment based on the comparison.

Clause 15. The wearable article according to clause 14, further including an inertial measurement unit (“IMU”) configured to monitor a position and orientation of the wearable article in three-dimensional space, and wherein, when executed by the processor, the visualization engine further causes the computing device to receive a second signal generated by the IMU, and wherein generation of the simulation is further based on the second signal received from the IMU.

Clause 16. The wearable article according to either clause 14 or 15, wherein, when executed by the processor, the visualization engine further causes the computing device to calibrate the second signal generated by the IMU based on the first signal generated by the first flexible circuit.

Clause 17. The wearable article according to any of clauses 14-16, further including a second flexible circuit, wherein the second flexible circuit includes a second trace including a deformable conductor, and wherein the second flexible circuit is positioned in a second location of interest on the wearable article.

Clause 18. The wearable article according to any of clauses 14-17, wherein the wearable article is a glove, and wherein the first location of interest includes a most proximal knuckle of a first finger of the glove.

Clause 19. The wearable article according to any of clauses 14-18, wherein the second location of interest includes an intermediate knuckle of the first finger.

Clause 20. A method of simulating a physical motion performed by a user via an avatar in a virtual environment, the method including developing a framework for electrical parameters generated by a plurality of flexible circuits of a wearable article, wherein the framework includes a plurality of scales that correlate the electrical parameters generated by each flexible circuit of the plurality of flexible circuits to physical conditions of each flexible circuit of the plurality of flexible circuits, receiving a plurality of signals generated in response to a user's motions while wearing the wearable article, wherein the plurality of signals correspond to electrical parameters generated by the plurality of flexible circuits of the wearable article, determining a first physical condition of a first flexible circuit of the plurality based on a first received signal of the plurality and the plurality of scales, determining a second physical condition of a second flexible circuit of the plurality based on a second received signal of the plurality and the plurality of scales, comparing the first physical condition to the second physical condition, and generating a simulation of the physical motion performed by a user via the avatar in the virtual environment based on the comparison.

Clause 21. A system configured to characterize a physical motion performed by a user, the system including a wearable article including a first flexible circuit, wherein the first flexible circuit includes a first trace including a deformable conductor, wherein the first flexible circuit is positioned in a first location of interest on the wearable article; and a computing device including a processor and a memory configured to store instructions that, when executed by the processor, cause the processor to receive a first signal generated by the first flexible circuit, determine a first electrical parameter based on the first signal, determine a physical condition of the first flexible circuit based on the first electrical parameter, compare the physical condition of the first flexible circuit to previously determined physical conditions associated with the wearable article, and characterize the physical motion performed by the user based on the comparison.

Clause 22. The system according to clause 21, wherein the wearable article further includes an inertial measurement unit (“IMU”) configured to monitor a position and orientation of the wearable article in three-dimensional space, and wherein, when executed by the processor, the instructions further cause the computing device to receive a second signal generated by the IMU, and wherein characterization of the physical motion is further based on the second signal received from the IMU.

Clause 23. The system according to either of clauses 21 or 22, wherein, when executed by the processor, the instructions further cause the computing device to calibrate the second signal generated by the IMU based on the first signal generated by the first flexible circuit.

Clause 24. The system according to any of clauses 21-23, wherein the computing device is positioned remotely relative to the wearable article.

Clause 25. The system according to any of clauses 21-24, wherein the wearable article further includes a transceiver configured to transmit signals to and from the computing device.

Clause 26. The system according to any of clauses 21-25, further including an electronic component including a power source configured to provide electrical power to the first flexible circuit, and wherein the wearable article further includes a mechanical component configured to selectively receive the electronic component.

Clause 27. The system according to any of clauses 21-26, wherein the electronic component further includes a memory configured to store data associated with the first signal generated by the first flexible circuit.

Clause 28. The system according to any of clauses 21-27, wherein the wearable article further includes a second flexible circuit, wherein the second flexible circuit includes a second trace including a deformable conductor, and wherein the second flexible circuit is positioned in a second location of interest on the wearable article.

Clause 29. The system according to any of clauses 21-28, wherein the wearable article is a glove, and wherein the first location of interest includes a most proximal knuckle of a first finger of the glove.

Clause 30. The system according to any of clauses 21-29, wherein the second location of interest includes an intermediate knuckle of the first finger.

Clause 31. The system according to any of clauses 21-30, wherein the second location of interest further includes a most distal knuckle of the first finger.

Clause 32. The system according to any of clauses 21-31, wherein the second flexible circuit traverses around the first flexible circuit.

Clause 33. The system according to any of clauses 21-32, wherein the wearable article further includes a third flexible circuit, wherein the third flexible circuit includes a third trace including a deformable conductor, wherein the third flexible circuit is positioned in a third location of interest on the wearable article, and wherein the third location of interest includes a second finger of the glove.

All patents, patent applications, publications, or other disclosure material mentioned herein are hereby incorporated by reference in their entireties as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present invention has been described with reference to various exemplary and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications, or combinations of any of the exemplary aspects may be made without departing from the scope of the invention. In addition, persons skilled in the art will recognize, or be able to ascertain using no more than routine experimentation, many equivalents to the various aspects of the invention described herein upon review of this specification. Thus, the invention is not limited by the description of the various aspects, but rather by the claims.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and that in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although claim recitations are presented in (a) sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures, or characteristics may be combined in any suitable manner in one or more aspects.

As used herein, the singular forms of “a,” “an,” and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise expressly stated.

The terms “about” or “approximately” as used in the present disclosure, unless otherwise specified, mean an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 200%, 105%, 100%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all sub-ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials are not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein, will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

Instructions used to program logic to perform various disclosed aspects can be stored within a memory in the system, such as dynamic random access memory (DRAM), cache, flash memory, or other storage. Furthermore, the instructions can be distributed via a network or by way of other computer-readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, any reference to a processor or microprocessor can be substituted for any “control circuit,” which may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein, “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets, and/or data recorded on non-transitory computer readable storage medium. Firmware may be embodied as code, instructions or instruction sets, and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission, or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

What is claimed is:

1. A system configured to simulate a physical motion performed by a user via an avatar in a virtual environment, the system comprising:
a wearable article comprising a first flexible circuit, wherein the first flexible circuit comprises a first trace comprising a deformable conductor, wherein the first flexible circuit is positioned in a first location of interest on the wearable article; and
a computing device comprising a processor and a memory configured to store a visualization engine that, when executed by the processor, causes the processor to:
receive a first signal generated by the first flexible circuit;
determine a first electrical parameter based on the first signal;
scale the first electrical parameter based on a predetermined simulation framework of the visualization engine, wherein scaling the first electrical parameter corresponds to a physical condition of the first flexible circuit;
compare the physical condition of the first flexible circuit to previously determined physical conditions associated with the wearable article; and
generate a simulation of the physical motion performed by a user via the avatar in the virtual environment based on the comparison.

2. The system of claim 1, wherein the wearable article further comprises an inertial measurement unit (“IMU”) configured to monitor a position and orientation of the wearable article in three-dimensional space, and wherein, when executed by the processor, the visualization engine further causes the computing device to:
receive a second signal generated by the IMU; and
wherein generation of the simulation is further based on the second signal received from the IMU.

3. The system of claim 2, wherein, when executed by the processor, the visualization engine further causes the computing device to:
calibrate the second signal generated by the IMU based on the first signal generated by the first flexible circuit.

4. The system of claim 1, wherein the computing device is positioned remotely relative to the wearable article.

5. The system of claim 4, wherein the wearable article further comprises a transceiver configured to transmit signals to and from the computing device.

6. The system of claim 1, further comprising an electronic component comprising a power source configured to provide electrical power to the first flexible circuit, and wherein the wearable article further comprises a mechanical component configured to selectively receive the electronic component.

7. The system of claim 6, wherein the electronic component further comprises a memory configured to store data associated with the first signal generated by the first flexible circuit.

8. The system of claim 1, wherein the wearable article further comprises a second flexible circuit, wherein the second flexible circuit comprises a second trace comprising a deformable conductor, and wherein the second flexible circuit is positioned in a second location of interest on the wearable article.

9. The system of claim 8, wherein the wearable article is a glove, and wherein the first location of interest comprises a most proximal knuckle of a first finger of the glove.

10. The system of claim 9, wherein the second location of interest comprises an intermediate knuckle of the first finger.

11. The system of claim 10, wherein the second location of interest further comprises a most distal knuckle of the first finger.

12. The system of claim 11, wherein the second flexible circuit traverses around the first flexible circuit.

13. The system of claim 9, wherein the wearable article further comprises a third flexible circuit, wherein the third flexible circuit comprises a third trace comprising a deformable conductor, wherein the third flexible circuit is positioned in a third location of interest on the wearable article, and wherein the third location of interest comprises a second finger of the glove.

14. A wearable article configured to simulate a physical motion performed by a user via an avatar in a virtual environment, the wearable article comprising:
a first flexible circuit, wherein the first flexible circuit comprises a first trace comprising a deformable conductor, wherein the first flexible circuit is positioned in a first location of interest on the wearable article; and
a circuit configured to communicably couple the first flexible circuit to a computing device comprising a processor and a memory configured to store a visualization engine that, when executed by the processor, causes the processor to:
receive a first signal generated by the first flexible circuit;
determine a first electrical parameter based on the first signal;
scale the first electrical parameter based on a predetermined simulation framework of the visualization engine, wherein scaling the first electrical parameter corresponds to a physical condition of the first flexible circuit;
compare the physical condition of the first flexible circuit to previously determined physical conditions associated with the wearable article; and
generate a simulation of the physical motion performed by a user via the avatar in the virtual environment based on the comparison.

15. The wearable article of claim 14, further comprising an inertial measurement unit (“IMU”) configured to monitor a position and orientation of the wearable article in three-dimensional space, and wherein, when executed by the processor, the visualization engine further causes the computing device to:
receive a second signal generated by the IMU; and
wherein generation of the simulation is further based on the second signal received from the IMU.

16. The wearable article of claim 15, wherein, when executed by the processor, the visualization engine further causes the computing device to:
calibrate the second signal generated by the IMU based on the first signal generated by the first flexible circuit.

17. The wearable article of claim 14, further comprising a second flexible circuit, wherein the second flexible circuit comprises a second trace comprising a deformable conductor, and wherein the second flexible circuit is positioned in a second location of interest on the wearable article.

18. The wearable article of claim 17, wherein the wearable article is a glove, and wherein the first location of interest comprises a most proximal knuckle of a first finger of the glove.

19. The wearable article of claim 18, wherein the second location of interest comprises an intermediate knuckle of the first finger.

20. A method of simulating of a physical motion performed by a user via an avatar in a virtual environment, the method comprising:
developing a framework for electrical parameters generated by a plurality of flexible circuits of a wearable article, wherein the framework comprises a plurality of scales that correlate the electrical parameters generated by each flexible circuit of the plurality of flexible circuits to a plurality of flexible to physical conditions of each flexible circuit of the plurality of flexible circuits;
receiving a plurality of signals generated in response to a user's motions while wearing the wearable article, wherein the plurality of signals correspond to electrical parameters generated by the plurality of flexible circuits of the wearable article;
determining a first physical condition of a first flexible circuit of the plurality based on a first received signal of the plurality and the plurality of scales;
determining a second physical condition of a second flexible circuit of the plurality based on a second received signal of the plurality and the plurality of scales;
comparing the first physical condition to the second physical condition; and
generating a simulation of the physical motion performed by a user via the avatar in the virtual environment based on the comparison.
